(19) United States
(12) Patent Application Publication
(10) Pub. No.: US 2024/0390907 A1
(43) Pub. Date: Nov. 28, 2024

(54) MICROFLUIDIC CELL DISPENSING PLATFORM

(71) Applicant: Hewlett-Packard Development Company, L.P., Spring, TX (US)

(72) Inventors: Viktor Shkolnikov, Palo Alto, CA (US); Alexander Govyadinov, Corvallis, OR (US)

(73) Assignee: Hewlett-Packard Development Company, L.P., Spring, TX (US)

(21) Appl. No.: 18/202,827

(22) Filed: May 26, 2023

(51) Int. Cl. B01L 3/00

(52) U.S. Cl. CPC B01L 3/502784; B01L 3/502715; B01L 3/52; B01L 2200/16; B01L 2300/0645

(57) ABSTRACT

Microfluidic systems are provided for the detection and analysis of cell systems that can include a set of reservoirs, the set of reservoirs including a reagent reservoir and an immiscible fluid reservoir, an incubation region having an inlet and an outlet, a set of independent fluidic networks, the set of independent fluidic networks including a first fluidic network and a second fluidic network that is not fluidically connected to the first fluidic network, wherein the first fluidic network (1) is fed by the set of reservoirs, (2) includes a droplet generator, and (3) connects to the inlet of the incubation region, and wherein the second fluidic network is connected to the outlet of the incubation region.

### BACKGROUND

There is an increased demand for developing custom proteins (and peptides) with an optimized function in chemical and pharmaceutical industries. Examples of such proteins range from antibodies with strong and specific binding to an antigen for therapy to lipase enzymes in laundry detergent that are superior at breaking down grease stains while surviving typical laundering and storage conditions and not breaking down fabric and dye. Currently, to find these proteins, researchers create a protein library, via cloning via a plasmid corresponding to specific mutations into specific organisms or direct mutagenesis, and subsequently subject the proteins to functional tests in individual chambers. From their synthesis the proteins are either all together in one chamber and are separated during functional characterization, or are synthesized in separate chambers. The production and testing of individual proteins often require purification and separation of individual protein variants, as well as testing their functions and is often laborious, costly, and limits the optimality that can be achieved. Thus, there is a need to produce libraries of protein variants that are pre-purified, separated by variant and produced in low cost manner.

### BRIEF DESCRIPTION OF THE DRAWINGS

FIG. 1 depicts cross-section views of examples of microfluidic systems according to various embodiments.

FIG. 2 to FIG. 8 depict top views of examples of microfluidic systems according to various embodiments.

FIG. 9 to FIG. 18 depict cross-sectional views of examples of microfluidic systems according to various embodiments.

FIG. 19 depicts an example flowchart of a method according to various embodiments.

FIG. 20 to FIG. 27 depict top views of examples of microfluidic systems according to various embodiments.

FIG. 28 depicts cross-sectional views of examples of fluid dispensing systems according to various embodiments.

FIG. 29 shows a schematic diagram of examples of microfluidic systems according to various embodiments.

FIG. 30 shows an example flowchart of a method according to various embodiments.

FIG. 31 depicts top views of examples of devices for synthesizing and dispensing a peptide in a droplet according to various embodiments.

FIG. 32 shows an example flowchart of a method according to various embodiments.

The foregoing and other features of the present disclosure will become apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the figures, may be arranged, substituted, combined, and designed in a wide variety of different configurations, all of which are explicitly contemplated and make part of this disclosure.

### Definitions

Unless defined otherwise, all technical and scientific terms used herein generally have the same meaning as commonly understood by one of ordinary skill in the art to which this technology belongs. Generally, the nomenclature used herein and the laboratory procedures in cell culture, molecular genetics, organic chemistry, analytical chemistry and nucleic acid chemistry and hybridization described below are those well-known and commonly employed in the art. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or illustrative language (e.g., “such as”) provided herein, is intended merely to better illuminate the embodiments and does not pose a limitation on the scope of the claims unless otherwise stated. No language in the specification should be construed as indicating any non-claimed element as essential.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without other input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

As used in this specification and the appended claims, the singular forms “a”, “an” and “the” include plural referents unless the content clearly dictates otherwise. For example, reference to “a cell” includes a combination of two or more cells, and the like. The use of the terms “a” and “an” and “the” and similar referents are to be construed to cover both the singular and the plural, unless otherwise indicated herein or clearly contradicted by context.

As used herein with respect to numerical ranges, the terms “approximately,” “about,” “substantially,” and similar terms will be understood by persons of ordinary skill in the art and will vary to some extent depending upon the context in which it is used. If there are uses of the terms that are not clear to persons of ordinary skill in the art, given the context in which it is used, the terms will be plus or minus 10% of the disclosed values. When “approximately,” “about,” “substantially,” and similar terms are applied to a structural feature (e.g., to describe its shape, size, orientation, direction, etc.), these terms are meant to cover minor variations in structure that may result from, for example, the manufacturing or assembly process and are intended to have a broad meaning in harmony with the common and accepted usage by those of ordinary skill in the art to which the subject matter of this disclosure pertains. Accordingly, these terms should be interpreted as indicating that insubstantial or inconsequential modifications or alterations of the subject matter described and claimed are considered to be within the scope of the disclosure as recited in the appended claims.

Thus, in some embodiments, the terms “approximately,” “about,” “substantially”, and similar terms are intended to have a broad meaning in harmony with the common and accepted usage by those of ordinary skill in the art to which the subject matter of this disclosure pertains. These terms are intended to allow a description of certain features described and claimed without restricting the scope of these features to the precise numerical ranges provided. Accordingly, these terms should be interpreted as indicating that insubstantial or inconsequential modifications or alterations of the subject matter described and claimed are considered to be within the scope of the disclosure as recited in the appended claims. With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances, where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.” Further, unless otherwise noted, the use of the words “approximate,” “about,” “around,” “substantially,” etc., mean plus or minus ten percent.

It should be noted that the terms “exemplary,” “example,” “potential,” and variations thereof, as used herein to describe various embodiments, are intended to indicate that such embodiments are possible examples, representations, or illustrations of possible embodiments (and such terms are not intended to connote that such embodiments are necessarily extraordinary or superlative examples). The term “example” as used throughout this application is by way of illustration, and not limitation.

The term “coupled” and variations thereof, as used herein, means the joining of two members directly or indirectly to one another. Such joining may be stationary (e.g., permanent or fixed) or moveable (e.g., removable or releasable). Such joining may be achieved with the two members coupled directly to each other, with the two members coupled to each other using a separate intervening member and any additional intermediate members coupled with one another, or with the two members coupled to each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic.

The term “or,” as used herein, is used in its inclusive sense (and not in its exclusive sense) so that when used to connect a list of elements, the term “or” means one, some, or all of the elements in the list. Conjunctive language such as the phrase “at least one of X, Y, and Z,” unless specifically stated otherwise, is understood to convey that an element may be either X, Y, Z; X and Y; X and Z; Y and Z; or X, Y, and Z (i.e., any combination of X, Y, and Z). Thus, such conjunctive language is not generally intended to imply that certain embodiments require at least one of X, at least one of Y, and at least one of Z to each be present, unless otherwise indicated.

References herein to the positions of elements (e.g., “top,” “cbottom,” “above,” “below,” “up,” “down”) may merely be used to describe the orientation of various elements as arranged in the Figures. It should be noted that the orientation of various elements may differ according to other potential embodiments, and that such variations are intended to be encompassed by the present disclosure.

As used herein, a “biological sample” may refer to whole cells and/or live cells and/or cell debris. The biological sample may contain (or be derived from) a bodily fluid that in one aspect is known to contain cells of a certain genotype or phenotype. A sample can also encompass samples originally isolated from a biological specimen but have been maintained or cultured in an artificial environment. Such cells can be useful as positive or negative controls. Non-limiting examples of a biological sample include amniotic fluid, aqueous humor, vitreous humor, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. In some embodiments, a biological sample comprises cell cultures, bodily fluids, cell cultures from bodily fluids. Bodily fluids can be obtained from a mammal e.g. a human for example by puncture, or other collecting or sampling procedures.

As used herein, the term “nucleic acid,” “polynucleotide,” or “oligonucleotide” generally refers to any polyribonucleotide or poly-deoxyribonucleotide, and includes unmodified RNA, unmodified DNA, modified RNA, and modified DNA. Polynucleotides include, without limitation, single- and double-stranded DNA and RNA polynucleotides. The term polynucleotide, as it is used herein, embraces chemically, enzymatically or metabolically modified forms of polynucleotides, as well as the naturally occurring chemical forms of DNA and RNA found in or characteristic of viruses and cells, including for example, simple (prokaryotic) and complex (eukaryotic) cells. A nucleic acid polynucleotide or oligonucleotide as described herein retains the ability to hybridize to its cognate complimentary strand. A nucleic acid sample will comprise nucleic acids that serve as templates for and/or substrates for a polymerization reaction. A polynucleotide useful for the methods described herein can be an isolated or purified polynucleotide; it can be an amplified polynucleotide in an amplification reaction, or a transcribed product from an in vitro transcription reaction. In some embodiments, different primary sequence could provide a variant of the protein.

Accordingly, as used herein, the term nucleic acid, polynucleotide or oligonucleotide also encompasses primers and probes, as well as oligonucleotide fragments, and is generic to polydeoxyribonucleotides (containing 2-deoxy-D-ribose), to polyribonucleotides (containing D-ribose), and to any other type of polynucleotide which is an N-glycoside of a purine or pyrimidine base, or modified purine or pyrimidine bases (including, but not limited to, abasic sites). There is no intended distinction in length between the term “nucleic acid,” “polynucleotide,” and “oligonucleotide,” and these terms are used interchangeably. These terms refer only to the primary structure of the molecule. An oligonucleotide is not necessarily physically derived from any existing or natural sequence, but can be generated in any manner, including chemical synthesis, DNA replication, DNA amplification, reverse transcription or any combination thereof.

As used herein, the terms the term “polynucleotide” is composed of a specific sequence of five nucleotide bases: adenine (A); cytosine (C); guanine (G); thymine (T); and uracil (U) for thymine when the polynucleotide is RNA. Thus, the term “polynucleotide sequence” is the alphabetical representation of a polynucleotide molecule. This alphabetical representation can be input into databases in a computer having a central processing unit and used for bioinformatics applications such as functional genomics and homology searching.

As used herein, the term “immiscible fluid” generally refers to any fluid that is immiscible with respect to another fluid. For example, an “immiscible fluid” may be an oil that is immiscible with respect to water or other aqueous solutions. As used herein, an “oil” is any chemical substance hydrophobic, such as a nonpolar chemical substance that is composed primarily of hydrocarbons and is lipophilic. Example immiscible fluids include, but are not limited to, silicone oil (e.g., 1 centistoke (cSt), 3 cSt, 5 cSt, or 10 cSt), mineral oil, kerosene, Isopar-G, Isopar-L, fluorinated hydrocarbons, polyphenyl-methylsiloxane, polydimethyl siloxane, hexadecane, tetradecane, octadecane, dodecane, isopar M, isopar L, Novec 7100, FC40, etc. Unless otherwise indicated, the term “oil” is used interchangeably with “immiscible fluid.”

As used herein, the terms “target nucleic acid,” “target RNA,” “target DNA,” “target oligonucleotide,” and “target polynucleotide,” refer to a nucleic acid of interest, e.g., a nucleic acid of a particular nucleotide sequence one wishes to amplify, detect and/or quantify in a sample using the approaches described herein. The target polynucleotide can be obtained from any source, and can comprise any number of different compositional components. For example, the target can be nucleic acid (e.g. DNA or RNA), transfer RNA, sRNA, and can comprise nucleic acid analogs or other nucleic acid mimic. The target can be methylated, non-methylated, or both. The target can be bisulfate-treated and non-methylated cytosines converted to uracil. Further, it will be appreciated that “target polynucleotide” can refer to the target polynucleotide itself, as well as surrogates thereof, for example amplification products, and native sequences. In some embodiments, the target polynucleotide is a nucleic acid sequence comprising a rare mutation. The terms can refer to a single-stranded or double-stranded polynucleotide molecule (e.g., RNA, DNA, as the case may be), or a specific strand thereof, to which, for example, an oligonucleotide primer that is “specific for” the target nucleic acid anneals or hybridizes. A target nucleic acid as used herein has at least a portion of sequence that is complementary to a target-specific oligonucleotide molecule, such as hairpin barcode primer.

A translatable target nucleotide sequence (e.g., a target ribonucleotide sequence) sometimes encodes a peptide, polypeptide or protein, which are sometimes referred to herein as “target peptides,” “target polypeptides” or “target proteins.” Any peptides, polypeptides or proteins may be encoded by a target nucleotide sequence and may be selected by a person of ordinary skill in the art. Representative proteins include antibodies, enzymes, serum proteins (e.g., albumin), hormones (e.g., growth hormone, erythropoietin, insulin, etc.), cytokines, etc., and include both naturally occurring and exogenously expressed polypeptides.

As used herein, the term “multiplex amplification” refers to amplification of multiple different target nucleic acid sequences in the same reaction (see, e.g., PCR PRIMER, A LABORATORY MANUAL (Dieffenbach, ed. 1995) Cold Spring Harbor Press, pages 157-171). “Multiplex amplification,” as used herein, refers to amplification of at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 30 or more targets, e.g., at least 50, at least 100, at least 250, at least 500, at least 750, at least 1000, at least 5000 or more, targets.

As used herein, the term “comprising” or “comprises” is used in reference to compositions, methods, and respective component(s) thereof, that are useful to an embodiment, yet open to the inclusion of unspecified elements, whether useful or not.

As used herein, the term “consisting essentially of” refers to those elements for a given embodiment. The term permits the presence of elements that do not materially affect the basic and novel or functional characteristic(s) of that embodiment of the disclosure.

The term “protein”, “peptide” and “polypeptide” are used interchangeably and in their broadest sense to refer to a compound of two or more subunit amino acids, amino acid analogs or peptidomimetics. The subunits may be linked by peptide bonds. In another embodiment, the subunit may be linked by other bonds, e.g., ester, ether, etc. A protein or peptide must contain at least two amino acids and no limitation is placed on the maximum number of amino acids which may comprise a protein's or peptide's sequence. As used herein the term “amino acid” refers to either natural and/or unnatural or synthetic amino acids, including glycine and both the D and L optical isomers, amino acid analogs and peptidomimetics. Proteins and polypeptides are known to function in biological systems as for examples, antigens, cytokines, cell markers, receptors, transcription factors, and the like.

As used herein, the terms “antigen” refers to all, part, fragment, or segment of a protein, polypeptide or molecule that can induce an immune response in a subject or an expansion of an immune cell, preferably a T or B cell.

As used herein, the terms “isolated” means separated from constituents, cellular and otherwise, which the polynucleotide, peptide, polypeptide, protein, antibody, or fragment(s) thereof, are normally associated with in nature. For example, with respect to a polynucleotide, an isolated polynucleotide is one that is separated from the 5′ and 3′ sequences with which it is normally associated in the chromosome. As is apparent to those of skill in the art, a non-naturally occurring polynucleotide, peptide, polypeptide, protein, antibody, or fragment(s) thereof, does not require “isolation” to distinguish it from its naturally occurring counterpart. In addition, a “concentrated” “separated,” or “diluted” polynucleotide, peptide, polypeptide, protein, antibody, or fragment(s) thereof, is distinguishable from its naturally occurring counterpart in that the concentration or number of molecules per volume is greater than “concentrated” or less than “separated” than that of its naturally occurring counterpart. A polynucleotide, peptide, polypeptide, protein, antibody, or fragment(s) thereof, which differs from the naturally occurring counterpart in its primary sequence or, for example, by its glycosylation pattern, need not be present in its isolated form since it is distinguishable from its naturally occurring counterpart by its primary sequence, or alternatively, by another characteristic such as its glycosylation pattern. A mammalian cell, such as T-cell, is isolated if it is removed from the anatomical site in which it is found in an organism.

Ins some embodiments, the term “isolated” with respect to nucleic acids, such as DNA or RNA, can refer to molecules separated from other DNAs or RNAs, respectively, that are present in the natural source of the macromolecule as well as polypeptides. The term “isolated” is also used herein to refer to polynucleotides, polypeptides, and proteins that are isolated from other cellular proteins and is meant to encompass both purified and recombinant polypeptides.

As used herein, the term “sequence identity” refers to the similarity between two or more nucleic acid sequences, or two or more amino acid sequences. Sequence identity can be measured in terms of percentage identity (e.g., 80%, 85%, 90%, or 95%). The higher the percentage, the more identical the sequences are. Homologs or orthologs of nucleic acid or amino acid sequences possess a relatively high degree of sequence identity/similarity when aligned using standard methods. Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations. The NCBI Basic Local Alignment Search Tool (BLAST) (Altschul et al., J. Mol. Biol. 215:403-10, 1990) is available from several sources, including the National Center for Biological Information (NCBI, National Library of Medicine, Building 38A, Room 8N805, Bethesda, Md. 20894) and on the Internet, for use in connection with the sequence analysis programs blastp, blastn, blastx, tblastn, and tblastx. Blastn is used to compare nucleic acid sequences, while blastp is used to compare amino acid sequences. Additional information can be found at the NCBI web site.

In some embodiments, the alignment program is BLASTN or BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST.

Once aligned, the number of matches is determined by counting the number of positions where an identical nucleotide or amino acid residue is presented in both sequences. The percent sequence identity is determined by dividing the number of matches either by the length of the sequence set forth in the identified sequence, or by an articulated length (such as 100 consecutive nucleotides or amino acid residues from a sequence set forth in an identified sequence), followed by multiplying the resulting value by 100. For example, a nucleic acid sequence that has 1166 matches when aligned with a test sequence having 1554 nucleotides is 75.0 percent identical to the test sequence (1166±1554*100=75.0). The percent sequence identity value is rounded to the nearest tenth. For example, 75.11, 75.12, 75.13, and 75.14 are rounded down to 75.1, while 75.15, 75.16, 75.17, 75.18, and 75.19 are rounded up to 75.2. The length value will always be an integer. In another example, a target sequence containing a 20-nucleotide region that aligns with 20 consecutive nucleotides from an identified sequence as follows contains a region that shares 75 percent sequence identity to that identified sequence (e.g., 15±20*100=75).

As used herein, the term “magnetic bead” refers to a nanometer-micrometer sized bead that can 1 bind to or complex to the target protein or fragment thereof that is of interest if present in the sample when functionalized, and in some aspects, isolate a target protein or fragment of interest. As used herein, a “target protein” or protein of interest includes target fragments of fragments of interest unless otherwise specified. The size of a magnetic bead varies, ranging from 10 nm to 5 μm. For example, the size of a magnetic bead may be 100 nm, 300 nm, 500 nm, 700 nm, or 1 μm. A magnetic bead may include a magnetic core, a surface coating, and specific binding ligands at the surface. Various materials may be used for magnetic beads. The core may include pure metals such as Co, Fe, and Ni or their oxides, and/or transition-metal-doped oxides and metal alloys, including CoPt₃, FeCo, and FePt. For example, the core may include iron oxides such as magnetite (Fe₃O₄) and maghemite (γ-Fe₂O₃). The surface coating may be formed by non-polymeric stabilization or polymeric stabilization. Example non-polymeric stabilizers include alkanesulphonic and alkanephosphonic acids, phosphonates, oleic acid, lactobionic acid, lauric acid, etc. Example polymeric stabilizers include alginate, chitosan, dextran, polyethylene glycol, polystyrene, polyvinyl alcohol, pullulan, polyethylene imine, etc.

As is apparent to the skilled artisan, the magnetic bead must be functionalized to contain a binding partner to the bind to, or complex with the protein or polypeptide of interest. For example, if the target protein or polypeptide is a cell surface receptor, the magnetic bead is functionalized with the receptor's ligand. The number, orientation and density of the functionalized peptides or proteins on the beads will vary with the purpose and limitations of the target to be detected, i.e., from about 0.05 peptide or protein molecules per 100 nm² of surface area of the magnetic bead, to about 30/100 nm², or alternatively from 0.1/100 nm² to about 25/100 nm², or alternatively from about 0.3/100 nm² to about 25/100 nm², or alternatively from about 0.4/100 nm² to about 25/100 nm², or alternatively from about 0.5/100 nm² to about 20/100 nm², or alternatively from 0.6/100 nm² to about 20 I/100 nm², or alternatively from about 1.0/100 nm² to about 20/100 nm², or alternatively from about 5.0/100 nm² to about 20 I/100 nm², or alternatively from about 10.0/100 nm² to about 20/100 nm², or alternatively from about 15/100 nm² to about 20/100 nm², or alternatively at least about 0.5, or alternatively at least about 1.0, or alternatively at least about 5.0, or alternatively at least about 10.0, or alternatively at least about 15.0 I1100 nm², the nm² surface area of the magnetic bead. Methods to functionalize magnetic beads are known in the art or are commercially available, see, e.g., https://www.lifetein.com/peptode-product/amineactivated-peptode-conjugate-magnetic-beads-p-3647.html (last accessed on May 8, 2023) and Whiteaker et al. (2007) Anal Biochem, March 1:362(1):44-54 and Andrews et al. (2017) Analyst December 18: 143(1):1330140. For example, functionalization of magnetic beads can occur via amine or carboxyl groups on the surfaces. For example, amine groups of antibodies or other proteins can be coupled to carboxyl groups on the surface of magnetic beads via ethyl(dimethylaminopropyl) carbodiimide/N-hydroxysuccinimide (EDC/NHS) chemistry. In some embodiments, the bead can be functionalized with several targets. In some embodiments, functionalization may differ for example, depending on the molecule size and the interaction between it and the binding partner. In some embodiments, the surface concentration of the target may be the highest concentration where steric hindrance, and/or electrostatic repulsion to binding is not present.

As used herein, the term “dispense” refers to or includes the release of a specific volume of a fluid containing the synthesized peptide as a droplet.

As used herein, the term “Cell-free Protein Synthesis” or “CFPS” refers to or includes peptide synthesis, including translation and optionally transcription in vitro without a living cell. CFPS reagents have compositions that promote or enable such synthesis. CFPS systems may be available as ready-made kits, such as: E. coli (e.g., New England Biolabs, Promega, Bioneer, Qiagen, Arbor Biosciences, ThermoFisher, Creative Biolabs), rabbit reticulocyte (e.g., Promega, Creative Biolabs), wheat germ (e.g., Promega, Creative Biolabs), Leishmania tarentolae (e.g., Jena Bioscience), insect (e.g., Qiagen, Creative Biolabs), Chinese hamster ovary (e.g., Creative Biolabs), HeLa (e.g., ThermoFisher, Creative Biolabs), and plant cells (e.g., LenioBio). The compositions of CFPS reagents can vary, but often includes a lysate of the organism in question, sometimes after certain proprietary purification.

As used herein, the term “support” means a solid or semisolid substrate to which something can be attached, such as a nucleic acid barcode, for example an origin-specific barcode. The attachment can be a removable attachment. Non-limiting examples of a support useful in the methods of the disclosure include a hydrogel, cell, bead, column, filter, slide surface, or interior wall of a discrete volume, such as a well in a microtiter plate, or vessel. In certain embodiments, the support is a hydrogel (such as a hydrogel bead) to which one or more origin-specific barcodes is coupled. A origin-specific barcodes reversibly coupled to a support can be detached from the support, for example enzymatic cleavage of a cleavage site on the origin-specific barcode. A support may be present in a discrete volume as set forth herein. In certain embodiments, the support is a hydrogel bead present in an emulsion droplet.

As used herein, the “microfluidic network” or channel thereof includes and/or refers to a path through which a fluid or semi-fluid may pass, which may allow volumes of fluid, such as microliter (L), nanoliter, picoliter, or femtoliter volumes, to be transported through the microfluidic device. A microfluidic network may be formed of an etched, microfabricated, and/or micromachined portion of the substrate. As used herein, “substrate” refers to or includes the underlying substance of the device. The substrate may be formed of any suitable material for microfluidics. Non-limiting examples of substrate material include silicon, glass, quartz, ceramics, or polymeric material, and combinations thereof, such as glass-epoxy laminates, glass/silicon wafers, etc.

As used herein, “magnet” refers to or includes a material to generate a magnetic field to trap magnetic particles in flow. Trapping refers to or includes capturing and immobilizing for a predetermined interval. The magnet includes conducting material, such as iron, nickel, cobalt, copper, gold, or aluminum, and mixtures or alloys thereof, or magnetically susceptible features to be magnetized with an external magnet.

The magnetic particle includes a material that responds to a magnetic field. The magnetic particle may be selected from nanoparticles and microparticles, such as nanospheres and microspheres. In some examples, the magnetic particle is a magnetic bead. Non-limiting examples of suitable magnetic particles include paramagnetic materials, superparamagnetic materials ferromagnetic materials, ferrimagnetic materials, and metamagnetic materials, such as iron, nickel, and cobalt, as well as metal oxides (e.g., Fe3O4, BaFe12O19, CoO, NiO, Mn2O3, Cr2O3, and CoMnP. In some examples, the surface of the magnetic particle is functionalized with a chemical moiety or functional group to permit attachment of the synthesized peptide or tag thereon.

As used herein, “tag” refers to or includes a polypeptide sequence genetically grafted onto a recombinant peptide. Tags can be encoded by the peptide template for various purposes including detecting, isolating and purifying the synthesized peptide. The peptide sequence of the tag can vary from about 10 amino acids to greater than 50 amino acids. Non-limiting examples of tags that can be encoded by the template include a FLAG-tag, a HA-tag, a His-tag, a Myc-tag, a Strep-tag, a TC tag, a VS tag, an NE-tag, an S-tag, an SBP-tag, a Spot-tag, an Isopeptag, a SpyTag, a BCCP-tag, a GST-tag, a GFP-tag, a HaloTag, a SNAP-tag, a CLIP-tag, an HUH-tag, an MBP-tag, and a TXN-tag.

In some examples, the droplet ejector comprises a thermal resistor. As used herein, “thermal resistor” refers to a heater that is capable of rapid, local superheating of a small volume of liquid (sample fluid, reporter fluid, etc.).

In some examples, the microfluidic network comprises a wash fluid channel in fluidic communication with the microfluidic channel upstream of the region, wherein the wash fluid channel comprises a constriction and a resistor to pump wash fluid through the constriction. As used herein, “wash fluid” refers to a buffer suitable for use with peptide-magnetic particle complexes, such as buffered saline optionally including a detergent.

In some examples, the microfluidic network comprises a dilution fluid channel in fluidic communication with the microfluidic channel at a location proximate the droplet ejector. As used herein, “dilution fluid channel” refers to or includes a path through which a volume of reporter fluid may flow to the microfluidic channel and dilute the immunoreactive magnetic particles suspended therein.

In some examples, the substrate comprises a resistor to agitate immunoreactive magnetic particle in the region of the microfluidic channel adjacent to the magnet. As used herein, “a resistor to agitate” refers to or includes a fluid driver to selectively agitate fluid in the region and thereby agitate the immunoreactive magnetic particles to facilitate release from the magnetic field of the magnet. The resistor may include any device to move fluid within microfluidic channel, such as a micromixer, piezoelectric pump or a thermal micro-pump or a thin film resistor that is integrated with the substrate.

### Microfluidic Systems

There has been a growing interest in single cell analysis in the biotech and especially pharmaceutical space. This trend has been fueled by both the realization that cells are not homogeneous, that selecting individual cells on their output is useful, and can reduce the cost of downstream analysis, such as for cell genomics, transcriptomics, and proteomics. These cell analysis assays may require individual cells to be encapsulated in a droplet, often with other reagents. The cells are then incubated in a droplet for a significant period of time to generate a product, often in response to the presence of the stimuli or the other reagents in the droplet. The droplets are then sorted according to the result of the assay, before being delivered to the downstream assay. Sorting is required, since while the costs of downstream assays have decreased, the cost still remains high (e.g. ˜1 k per transcriptome), hence brute force screening of the entire droplet pool (typically >10,000 cells) still remains prohibitive. As the number of droplets generated is large (>10,000, often >10⁶), the droplets traditionally either need to occupy a large area on the chip in a droplet storage or need to be dispensed off chip in a storage region, and then reintroduced back into a chip for sorting. The first approach increases chip area, with the storage area being 10-100× the active area of the chip, drastically increasing chip cost. Meanwhile the active fluidic area can be <1×1 mm. The second approach increases the chances for contamination, as the cells typically have to leave the sterile cartridge. This disclosure overcomes the limitations of these prior methods and provides, in various embodiments, a solution for the problem of encapsulating the cell in a droplet, incubating this in a low cost storage region, and then further processing it, without contamination.

Several microfluidic systems are provided herein. Microfluidic systems may include a channel and a set of reservoirs feeding the channel. The microfluidic systems may include an incubation region for cell incubation. The microfluidic system may eject at least a portion of the incubated cell for downstream processing.

The disclosure includes various embodiments of a system for high throughput cell screening and for selecting cells that are producing a specific product. In one aspect, the system takes a cell suspension with each cell having a nucleic acid, for example, encoding a different product. The system includes a fluidic layer (e.g., formed of SU8) that forms one or more fluidic networks. In the fluidic networks, the system can package a cell with a droplet such that each droplet contains a single cell. In some embodiments, the system can package each cell one at a time, or can package multiple cells with a respective droplet a time. In some embodiments, the cells (or molecules of interest) may be detected by a pair of electrodes with an impedance measurement system, before packaging the cells in droplets, to ensure high droplet utilization (e.g., relative to limiting dilution). The cells can be packaged with reagents necessary for detecting, identifying and/or measuring the molecule or product of interest (e.g., a cell, a particle in the cell or the droplet, etc.), specifically for homogeneous proximity assays). The droplets may then be ejected into a droplet storage reservoir for incubation. Such a chamber can incubate 10⁶-10⁹ droplets, and the same order of cells. With an approach of storing droplets in the fluidic layer (e.g., formed of SU8), storing 10⁸ of 10 μm×10 μm×10 μm droplet would take 100 cm² (e.g., a 100×100 mm) substrate. With the system described herein, the same number of droplets can be contained in a 5 mm×5 mm×5 mm chamber. After this incubation period, the droplets may be pulled (or otherwise introduced) into a second fluidic network, where the droplets can be sorted according to the presence of the desired product or molecule contained in each discreet droplet. The sorting mechanism may be an optical one, for example, with an excitation illumination and a detection collection wavelength. The droplets containing the desired molecule or product may be ejected into specific wells of a microtiter plate or other appropriate surface, for example, a single droplet dedicated to a single well. In some embodiments, the sorting mechanism may include detecting a lack of a product. For example, the sorting mechanism may determine that there is no certain product based on an optical and/or electrical measurement. In one aspect, these are delivered for downstream processing. The droplets that do not contain the desired product or molecule may be ejected into “junk” wells.

In one aspect, the disclosure includes various embodiments of a an ink jet (thermal ink jet (TIJ), piezo ink jet (PIJ), etc.) actuator-based integrated microfluidic device for dispensing a protein library (e.g., one protein variant at a time), into a multiwell plate for downstream analysis. Other jetting mechanisms may be used, such as electro spraying technology (e.g., for ejection of droplets out of a microfluidic chip). The device may include an input reservoir, an immiscible fluid reservoir (also referred to as oil reservoir), a channel including an incubation region, downstream of which are magnetic actuation regions, and side channels with, for example, TIJ resistors to receive waste product. The channel may form a droplet generator, transport the droplets downstream, and/or perform magnetic operations on the train of droplets. The input reservoir may include a nucleic acid template that can encode a target protein or fragment thereof, magnetic beads with affinity to the target protein or fragment thereof, and/or reagents for cell-free translation (and/or transcription) of the nucleic acid template. The nucleic acid template may be at a low concentration (limiting dilution concentration) such that at least about 37% of the droplets contain 1 nucleic acid template, and less than 5% of the droplets contain two or more templates. In some embodiments, each of the templates may be the same or different from each other. In one aspect, each template may encode for a different variant of the target protein. The device may combine the template with the cell-free translation (and/or transcription) reagents to produce a target protein or a fragment thereof, and then purifies the target protein or fragment using magnetic beads with affinity to the target protein or fragment thereof and TIJ driven droplet manipulation (or, e.g., other ink jet actuation), and elute the protein or fragment bound to the affinity tagged magnetic bead and eject it into a multiwell plate.

The disclosure includes various embodiments of a microfluidic system for high throughput cell screening and for selecting cells that are producing a specific product, e.g. the target protein or fragment thereof. The system may include at least one reagent reservoir (CFPS or supply) and an oil reservoir connected to a droplet generator, which in turn may be connected to a large volume incubation reservoir. The incubation reservoir may be capable of dispensing individual droplets containing reagents into another on-chip microfluidic network. The system may include an active silicon layer with fluid (e.g., “ink”) feed holes connecting the reservoirs with a SU8 fluidic network. The system may include several reagent reservoirs, several incubation reservoirs, droplet sensing electrodes, and fluorescent or other optics for characterizing the content of droplets before dispensing of droplets for downstream processing.

In some examples, the disclosure relates to a microfluidic system with a set of reservoirs, the set of reservoirs including a reagent reservoir and an immiscible fluid reservoir; an incubation region having an inlet and an outlet; a set of independent fluidic networks, the set of independent fluidic networks including a first fluidic network and a second fluidic network that is not fluidically connected to the first fluidic network, wherein the first fluidic network (1) is fed by the set of reservoirs, (2) includes a droplet generator, and (3) connects to the inlet of the incubation region, and wherein the second fluidic network is connected to the outlet of the incubation region.

In some examples, the disclosure relates to a microfluidic system with a channel; a set of reservoirs feeding the channel, the set of reservoirs including: an input reservoir including magnetic beads; and an oil reservoir; a droplet generator downstream of a junction between the channel and the input reservoir; an incubation region downstream of the junction; and downstream of the incubation region, one or more magnetic actuation regions, and one or more side channels.

### Methods for Isolating Target Proteins or Fragments Thereof

In some aspects, the disclosure relates to a method comprising, or consisting essentially of, or yet further consisting of: combining a set of inputs from one or more input reservoirs of a microfluidic device, the one or more input reservoirs (i) feeding into a main channel of the microfluidic device, and (ii) including nucleic acid (NA) templates, cell-free nucleic acid transcription and translation reagents, and functionalized magnetic beads, each NA template coding for a different variant of a protein of interest or a fragment thereof, encapsulating, using an immiscible fluid (e.g., an oil) from an immiscible fluid reservoir (e.g., oil reservoir) of the microfluidic device, the set of inputs into a series of droplets using a droplet generator of the microfluidic device such that each droplet in a subset of the series of droplets includes one or more, or alternatively only one NA template (e.g., multiple copies of a same NA having the same primary nucleotide sequence, only one copy of the NA, etc.); incubating, at an incubation zone of the main channel, the subset of droplets to synthesize target proteins or fragments thereof that can bind to the functionalized magnetic beads; passing, via the main channel, the incubated subset of droplets through a separation region of the microfluidic device to decouple the synthesized target proteins or fragments thereof from the functionalized magnetic beads; and removing or ejecting decoupled proteins out of the microfluidic device and into wells of a multiwell plate such that each well includes one product of the NA template, e.g., a single variant of the protein. Beads may be functionalized by, for example, binding proteins (e.g., ligands) to the beads to create bead-protein (fragment) conjugates when exposes to the target proteins or polypeptides.

In some examples, the disclosure relates to a device and its use, the device containing a microfluidic network supported and use thereof, the device including a main channel; a droplet ejector coupled to a distal end of the main channel to draw a first fluid through the main channel and dispense an eluted protein (or a fragment thereof; e.g., peptide) droplet; a first inlet to suspend a droplet of a second fluid in the first fluid flowing in the main channel, the second fluid including reagents (e.g., cell-free peptide synthesis (CFPS) reagents) and functionalized magnetic particles; an incubation zone to synthesize a peptide in the suspended droplet at a desired temperature; a first outlet fluidically downstream from the incubation zone and including a fluid actuator to draw a waste droplet including the CFPS reagents from the suspended droplet; a second inlet coupled to the main channel to merge an elution fluid droplet with the suspended droplet fluidically downstream from the first outlet; and a magnet disposed in the substrate to extract the eluted-peptide droplet from the suspended droplet.

In some examples, aspects of the disclosure relate to a method including: performing a series of cell-free protein (or a fragment thereof; e.g., peptide) synthesis reactions in particle-laden droplets flowing in a microfluidic network, wherein the microfluidic network includes: a proximal end receiving a carrier fluid, a distal end coupled to a droplet ejector, and an array of junctions between the proximal and distal ends; actuating the droplet ejector to flow the carrier fluid in the microfluidic network and suspend a reaction fluid into a plurality of droplets into the flowing carrier fluid at a first junction, the reaction fluid including cell-free peptide synthesis reagents (CFPS) including a set of nucleic acid templates each encoding a peptide, and a plurality of magnetic particles functionalized to capture a synthesized peptide, whereby a suspended droplet includes a subset of both the nucleic acid templates and the magnetic particles; flowing the suspended droplet through an incubation zone of the microfluidic network under conditions for CFPS; actuating an inertial pump proximate to a second junction to separate a reagent fluid droplet from the suspended droplet; merging an elution fluid droplet into the suspended droplet at a third junction fluidically downstream from the second junction to release synthesized peptides from the magnetic particles; activating a magnetic field to immobilize the magnetic particles; and actuating the droplet ejector to dispense the synthesized peptides at a pre-determined location.

In some examples, the disclosure relates to a system with a fluid supply assembly including: a first fluid reservoir to supply an encapsulating fluid (e.g., an oil or other immiscible fluid), a second fluid reservoir to supply a second fluid, wherein the second fluid includes cell-free protein (or peptide) synthesis (CFPS) reagents and magnetic particles functionalized to capture synthesized peptides; and a third fluid reservoir to supply an elution fluid; a dispenser assembly including: a microfluidic network supported by a substrate including a main channel in fluidic communication with the first fluid reservoir to flow the encapsulating fluid through a temperature controlled zone of the substrate downstream to a droplet ejector coupled to a distal end of the main channel; a plurality of inlets fluidically coupled to the fluid supply assembly and the main channel, wherein a first inlet suspends a droplet of the second fluid in the encapsulating fluid, and a second inlet disposed between the first inlet and the droplet ejector supplies the elution fluid to the suspended droplet; and a first outlet associated with a fluid actuator and a magnet to separate a waste droplet including the CFPS reagents from the suspended droplet; and a collection assembly to receive droplets dispensed by the droplet ejector.

In some examples, the disclosure relates to a system with a microfluidic network supported by a substrate including a main channel; a droplet ejector coupled to a distal end of the main channel; a first inlet to suspend a particle-laden droplet of a reaction fluid in a first fluid flowing in the main channel, the reaction fluid including cell-free protein (or peptide) synthesis (CFPS) reagents and functionalized magnetic particles; an incubation zone to synthesize a peptide in the particle-laden droplet; a first outlet fluidically downstream from the incubation zone and including a fluid actuator; a second inlet coupled to the main channel and disposed fluidically downstream from the first outlet; a magnet disposed in the substrate proximate to the droplet ejector, and a controller operatively connected to the incubation zone, the droplet ejector, the fluid actuator, and the magnet, wherein the controller includes a processing unit and a non-transient computer readable medium containing instructions that when executed, cause the processing unit to: control a temperature of the particle-laden droplet in the incubation zone; actuate the fluid actuator to remove a waste droplet including the CFPS reagents from the particle-laden droplet via the first outlet; apply a magnetic field to immobilize the particle-laden droplet; and actuate the droplet ejector to draw a first fluid through the main channel and suspend the particle-laden droplet via the first inlet; to draw an elution fluid into the main channel via the second inlet and merge the elution fluid and the particle-laden droplet; or to separate peptides from the immobilized particles and dispense the eluted peptides in a volume of fluid at a predetermined location.

The microfluidic systems disclosed herein allow for efficient creation and utilization of protein libraries. The microfluidic systems disclosed herein allow for lower requirements for reagents storage, and ability to ship reagents with microfluidic device (relative to protein synthesis via live organisms). In addition, the microfluidic systems disclosed herein allow for low cost production of protein libraries on demand and lower requirements for purification and cleaner resulting product as the system can produce only one product per droplet (relative to protein synthesis via live organisms). The microfluidic systems disclosed herein allow for lower cost devices, cell selection with long incubation times, reduction in chance of contamination, ability to process large numbers of cells (e.g., on the order of 10⁹ cells), and an ability to obtain cells with more optimal production of desired products (e.g., protein, polypeptide, antibody, metabolite, or an enzyme).

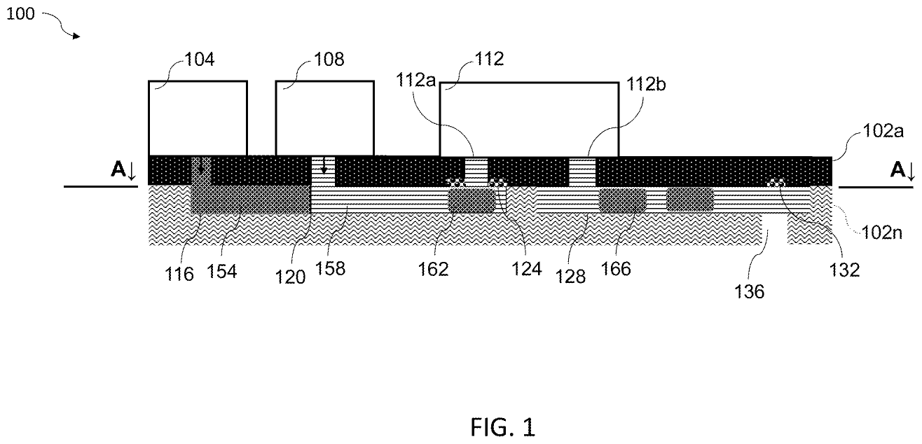

FIG. 1 shows cross-section views of examples of microfluidic systems 100 according to various embodiments. The microfluidic system 100 may include an active layer 102a, a network layer 102n, a reagent reservoir 104 (sometimes referred to as an input reservoir), an immiscible fluid reservoir 108 (sometimes referred to as an oil reservoir), an incubation reservoir 112, an inlet 112a and an outlet 112b of the incubation reservoir 112, a first fluidic network 116, a junction 120, a first fluid actuator 124, a second fluidic network 128, a second fluidic actuator 132, and an output 136. The microfluidic system 100 may include a reagent 154 (sometimes referred to as a cell), an immiscible fluid 158 (sometimes referred to as an oil), a droplet 162, and an incubated droplet 166.

The microfluidic system 100 may include a set of reservoirs, the set of reservoirs including a reagent reservoir and an immiscible fluid reservoir. The microfluidic system 100 may include an incubation region having an inlet and an outlet. The microfluidic system 100 may include a set of independent fluidic networks, the set of independent fluidic networks including a first fluidic network and a second fluidic network that is not fluidically connected to the first fluidic network, wherein the first fluidic network (1) may be fed by the set of reservoirs, (2) may include a droplet generator, and/or (3) may connect to the inlet of the incubation region, and wherein the second fluidic network may be connected to the outlet of the incubation region.

The active layer 102a is a layer to connect one or more fluidic networks in the network layer 102n (e.g., the first fluidic network 116, the second fluidic network 128, etc.) with one or more reservoirs (e.g., the reagent reservoir 104, the immiscible fluid reservoir 108, the incubation reservoir 112, etc.). The active layer 102a may be formed of silicon (e.g., active silicon layer). The active layer 102a may include a plurality of holes. The plurality of holes may include one or more feed holes (e.g., the inlet 112a, the outlet 112b, etc.). In some embodiments, as shown in FIG. 1, the active layer 102a may include one or more holes for each reservoir to fluidically connect the first fluidic network 116 and the second fluidic network 128 with the respective one or more reservoirs.

The reagent reservoir 104 (sometimes referred to as an input reservoir) can contain the reagent 154 and provide the reagent 154 to the first fluidic network 116. The reagent reservoir 104 may be a reservoir for cells. The reagent 154 may be or include, but not limited to reagents including a cell, cell-free translation (and/or transcription) reagents, nucleic acid templates, or magnetic beads with affinity to target protein or fragments thereof, etc. The nucleic acid template may be at a low concentration (limiting dilution concentration) such that about 37% of the droplets contain 1 template, and less than 5% of the droplets contain two or more templates. The reagent reservoir 104 may be disposed on the active layer 102a. In some embodiments, the reagent 154 may include a homogenous proximity assay, such as the Alpha Screen assay (commercially available assay) reagents. The reagent 154 for this assay may include a donor bead with affinity probes, and an acceptor beads with an affinity probes. When the correct product is present, the affinity probes on both beads can bind to the product, and so both beads can be forcing the beads to be in proximity to each other. The donor bead can then be excited by the incoming illumination, transfer energy to the acceptor bead when it is in proximity of the donor bead, and then the acceptor bead can emit photons which then be detected.

The immiscible fluid reservoir 108 (sometimes referred to as an oil reservoir) can contain the immiscible fluid 158. The immiscible fluid 158 may be or include, but not limited to oils, fluid that is less denser than the reagent 154, or fluid that is denser than the reagent 154, etc. The immiscible fluid reservoir 108 may be disposed on the active layer 102a. The immiscible fluid reservoir 108 can provide the immiscible fluid 158 to the first fluidic network 116. In some embodiments, the immiscible fluid reservoir 108 may be connected to a droplet generator or the junction 120.

The network layer 102n is a layer to form one or more fluidic networks (e.g., the first fluidic network 116, the second fluidic network 128, etc.). The network layer 102n may be formed of SU8. The network layer 102n and/or the fluidic networks formed therein (e.g., the first fluidic network 116, the second fluidic network 128, etc.) may include one or more holes that are connected to one or more reservoirs (e.g., the reagent reservoir 104, the immiscible fluid reservoir 108, the incubation reservoir 112, etc.) or outputs (e.g., the output 136). The first fluidic network 116 can be fed by the reagent reservoir 104. The first fluidic network 116 can communicate the received fluid (e.g., the reagent 154 fed by the reagent reservoir 104) through the junction 120. The first fluidic network 116 can be fed by the immiscible fluid reservoir 108. The first fluidic network 116 can communicate the received fluid (e.g., the immiscible fluid 158 fed by the immiscible fluid reservoir 108) through the inlet 112a.

The network layer 102n may include the junction 120 (or a droplet generator). The junction 120 or the droplet generator can encapsulate the reagent 154 into the droplet 162, or otherwise generate droplets based on the reagent 154 fed by the reagent reservoir 104. The junction 120 or such a droplet generator may be a droplet generating geometry to form the droplet 162 with a certain amount of the reagent 154 and encapsulate the droplet 162 with the immiscible fluid 158. In some embodiments, various geometric features may be used in the junction 120 and/or the first fluidic network 116 to generate droplets specific to the geometric features. For example, various sizes of channels at a droplet generator junction, in combination with various flow rates in the respective channels, can achieve a desired droplet generation. A large number of combinations work, and channel sizes and flow rates may be empirically tuned. In some embodiments, the junction 120 or such a droplet generator may be an ink jet based (e.g., a TIJ) dispenser jetting aqueous fluid (e.g., the reagent 154) into the immiscible fluid 158 or oil. In some embodiments, the junction 120 can split the reagent 154 into the droplet 162 enveloped by the immiscible fluid 158.

In the first fluidic network 116, the droplet 162 can move to the incubation region (e.g., adjacent to the incubation reservoir 112, the inlet 112a of the incubation reservoir 112, the first fluid actuator 124, etc.), the droplet 162 can be introduced into the incubation reservoir 112 through the inlet 112a by the first fluid actuator 124. The first fluidic network 116 and the incubation reservoir 112 may be fluidically connected via the inlet 112a. In some embodiments, the first fluidic network 116 may be connected to the inlet 112a of the incubation reservoir 112 via the first fluid actuator 124. For example, the first fluid actuator 124 provided adjacent to the inlet 112a can introduce the droplet 162 into the incubation reservoir 112.

The first fluid actuator 124 is to introduce the droplet 162 into the incubation reservoir 112. In some embodiments, the first fluid actuator 124 may be or include a TIJ nozzle/actuator or an inverted TIJ nozzle/actuator. In some embodiments, the inverted TIJ nozzle/actuator may act as a “pull pump” and drive the flow through the first fluidic network 116, operating a droplet generator.

In some embodiments, the fluid actuators described herein (e.g., the first fluid actuator 124, the second fluidic actuator 132, etc.) may be or include a piezoelectric inkjet (PIJ) actuator, and/or other actuation architectures. In some embodiments, a fluid actuator may be or include an inertial pump that includes a thermal actuator having a heating element (e.g., a thermal resistor) that may be heated to cause a bubble to form in a fluid proximate the heating element. In such examples, a surface of a heating element (having a surface area) may be proximate to a surface of a fluid channel in which the heating element is disposed such that fluid in the fluid channel may thermally interact with the heating element. In some embodiments, the heating element may include a thermal resistor with at least one passivation layer disposed on a heating surface such that fluid to be heated may contact a topmost surface of the at least one passivation layer. Formation and subsequent collapse of such bubble may generate flow of the fluid. In some embodiments, the fluid actuator(s) forming an inertial pump may include piezo-membrane based actuators, electrostatic membrane actuators, mechanical/impact driven membrane actuators, magnetostrictive drive actuators, electrochemical actuators, external laser actuators (e.g., that form a bubble through vaporization of a portion of the fluid with a laser beam), other such microdevices, or any combination thereof. In some embodiments, the fluid actuators described herein may displace fluid through movement of a membrane (such as a piezo-electric membrane) that generates compressive and tensile fluid displacements to thereby cause inertial fluid flow. In some embodiments, actuators and/or nozzles described herein may include a resistor. In some embodiments, such an actuator/nozzle may be, or may include, an inertial pump that includes a thermal actuator having a heating element (e.g., a thermal resistor) that may be heated to cause a bubble to form in a fluid proximate the heating element. In such examples, a surface of a heating element (having a surface area) may be proximate to a surface of a fluid channel in which the heating element is disposed such that fluid in the fluid channel may thermally interact with the heating element. In some embodiments, the heating element may include a thermal resistor with at least one passivation layer disposed on a heating surface such that fluid to be heated may contact a topmost surface of the at least one passivation layer. Formation and subsequent collapse of such bubble may generate flow of the fluid. In other examples, the fluid actuator(s) forming an inertial pump may include piezo-membrane based actuators, electrostatic membrane actuators, mechanical/impact driven membrane actuators, magnetostrictive drive actuators, electrochemical actuators, external laser actuators (e.g., that form a bubble through vaporization of a portion of the fluid with a laser beam), other such microdevices, or any combination thereof. In some implementations, the fluid actuators may displace fluid through movement of a membrane (such as a piezo-electric membrane) that generates compressive and tensile fluid displacements to thereby cause inertial fluid flow.

The incubation reservoir 112 may be a reservoir or an incubation chamber to perform various operations on the droplet 162 included therein. For example, in some embodiments, the incubation reservoir 112 can maintain the temperature and incubate at a temperature (e.g., 37° C.). For example, in some embodiments, the incubation reservoir 112 can have access to a gas exchange chamber and incubate at a certain environment (e.g., 5% CO₂ saturation). For example, the incubation reservoir 112 may incubate on the order of 24 hours. In some embodiments, the incubation reservoir 112 may include a receiving portion or part to pull the droplet 162, and/or an output portion or part to output the incubated droplet 166 into the second fluidic network 128.

In some embodiments, the incubation reservoir 112 may be a device that provides conditions to encourage or facilitate growth and/or maintenance of cell cultures. The incubation reservoir 112 may control and regulate such ambient conditions as temperature, humidity, gas composition of the atmospheric in a chamber in which cells are positioned (e.g., carbon dioxide, oxygen, and/or nitrogen content), ventilation, etc. An adjustable heater, for example, may be used to bring temperatures to 60 to 65° C. (140 to 150° F.), or may be as high as 100° C. A commonly-used temperature for mammalian cells is approximately 37° C. (99° F.), as such a temperature promotes growth of the cells. Some incubation reservoirs may have the ability to lower temperatures (e.g., via refrigeration or ventilation).

The incubation reservoir 112 may include the outlet 112b. The outlet 112b may be fluidically connected to the second fluidic network 128. Once the incubation is complete on the droplet 162, the incubated droplet 166 can be introduced into the second fluidic network 128 through the outlet 112b. Although not depicted, one or more fluid actuators may be disposed adjacent to the outlet 112b and may control the output of the incubated droplet 166.

The second fluidic network 128 is a fluidic channel independent from the first fluidic network 116. The incubated droplet 166 can be introduced into the second fluidic network 128, and can move through the output region adjacent to the second fluidic actuator 132 or the output 136.

The incubated droplet 166 can be ejected through the output 136. In some embodiments, the incubated droplet 166 may be ejected into wells of a microwell plate for downstream processing. In some embodiments, before the incubated droplet 166 is ejected, the incubated droplet 166 may be sorted according to presence of a product in the incubated droplet 166. According to the sorting, one or more of the incubated droplet 166 may be ejected into one or more specific wells of a microtiter plate. In some embodiments, the incubated droplet 166 may be ejected through the output 136 for downstream assays (e.g., proximity assays). In some embodiments, the incubation reservoir 112 may be capable of dispensing individual droplets containing the reagent 154 into another on-chip microfluidic network.

In some embodiments, the second fluidic actuator 132 may be provided adjacent to the output 136. In some embodiments, the second fluidic actuator 132 can control output of the incubated droplet 166. For example, the second fluidic actuator 132 may act as a valve, such that the incubated droplet 166 can be ejected through the output 136 when the second fluidic actuator 132 allows. The second fluid actuator may be a TIJ nozzle/actuator. The second fluidic actuator 132 can provide the incubated droplet 166 with a jet so as to eject the incubated droplet 166 through the output 136.

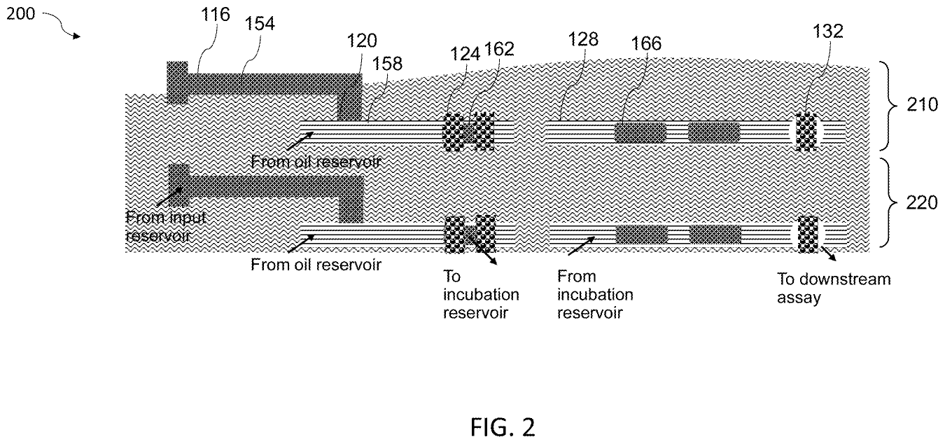

FIG. 2 shows top views of examples of microfluidic systems 200 according to various embodiments. In some embodiments, the microfluidic system 200 may include a plurality of sets of fluidic networks. For example, the microfluidic system 200 may include a first set of fluidic networks 210 and a second set of fluidic networks 220. In some embodiments, the first set of fluidic networks 210 and the second set of fluidic networks 220 may be substantially different. For example, the reagent 154 contained each in the first set of fluidic networks 210 and the second set of fluidic networks 220 may be different. For example, the incubating condition of each the first set of fluidic networks 210 and the second set of fluidic networks 220 may be different. For example, the sorting mechanism of each the first set of fluidic networks 210 and the second set of fluidic networks 220 may be different. In some embodiments, the first set of fluidic networks 210 and the second set of fluidic networks 220 may be substantially identical.

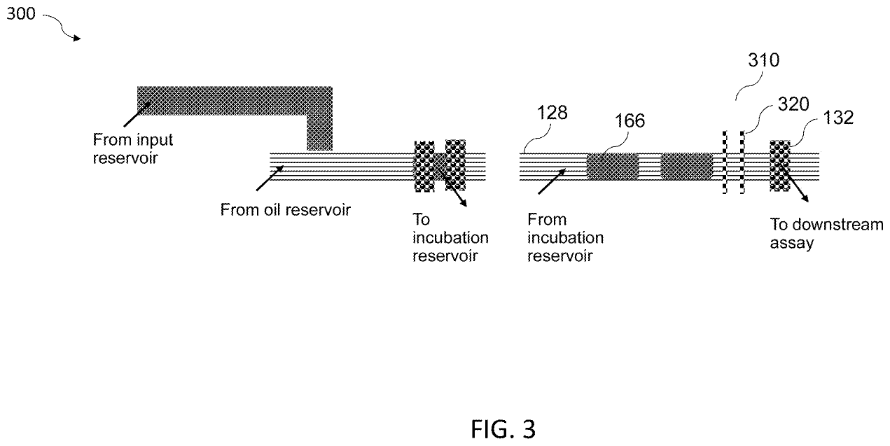

FIG. 3 shows top views of examples of microfluidic systems 300 according to various embodiments. The microfluidic system 300 includes a sensing region 310. The sensing region 310 may be disposed at the downstream side of the outlet 112b to detect one or more types of contents, particles, or cells, etc. The sensing region 310 may include an electrical detection system. For example, in some embodiments, the sensing region 310 may include impedance sensing electrodes 320. The electrical detection system can characterize the incubated droplets and sort the incubated droplets based on the characterization.

For example, after the incubation is complete, the incubated droplet 166 can be introduced into the second fluidic network 128. When the incubated droplet 166 passes through the sensing region 310, the impedance sensing electrodes 320 can measure an electrical property (e.g., impedance) of the incubated droplet 166, and detect a certain type of product(s) based on the measured property. In some embodiments, the impedance sensing electrodes 320 may measure the impedance for a broad spectrum of frequencies. In some embodiments, the impedance sensing electrodes 320 may measure the impedance for a particular set of frequencies. For example, the impedance sensing electrodes 320 may measure impedance at 10 kHz, 100 kHz and 1 MHz. Based on the change of impedance, measurements from the impedance sensing electrodes 320 can be used to detect the presence of a droplet. According to the detection, the incubated droplet 166 can be sorted. The sorted droplets may be ejected for downstream assays, for example ejected into one or more specific wells of a microtiter plate (e.g., a single droplet dedicated to a well), and then may be delivered to a user for downstream processing. In some embodiments, droplets that are not sorted may be ejected into junk wells. In some embodiments, the sorted droplets may be ejected for different downstream assays according to the detection.

In some embodiments, the second fluidic actuator 132 may operate based at least in part on a detection in the sensing region 310. The sensing region 310 may send a controller a signal associated with a first detection from a first incubated droplet, and the controller may determine whether to activate the second fluidic actuator 132 to eject the first incubated droplet that caused the first detection. For example, the second fluidic actuator 132 may eject the incubated droplet 166 when the sensing region 310 and/or the impedance sensing electrodes 320 detect a pre-determined type of content.

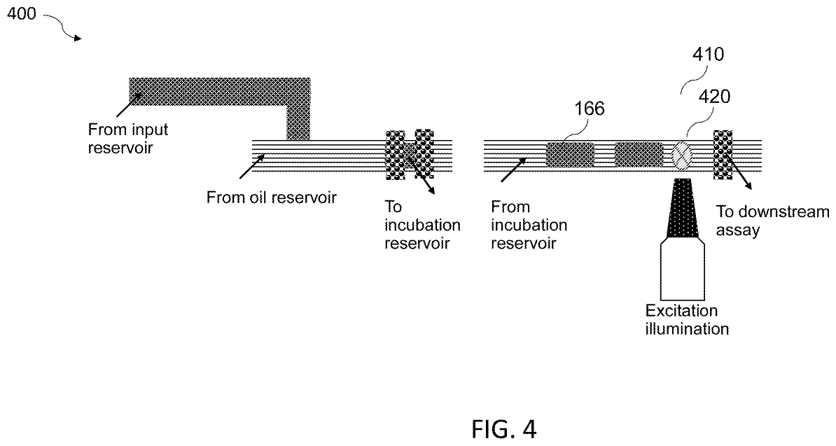

FIG. 4 shows top views of examples of microfluidic systems 400 according to various embodiments. In some embodiments, a sensing region 410 of the microfluidic system 400 may include one or more components according to various embodiments. For example, such components may be disposed at the downstream side of the outlet 112b. In some embodiments, as opposed to the microfluidic system 300, the microfluidic system 400 includes one or more optical components 420. The one or more optical components 420 may be to characterize content of droplets and/or to sort based on the characterization or optical detection.

In some embodiments, at least one of the one or more optical components 420 can collect light reflected from, transmitted from, or otherwise interacted with the incubated droplet 166. In some embodiments, at least one of the one or more optical components 420 can detect a certain wavelength and/or determine optical properties of the incubated droplet 166 passing through the sensing region 410. For example, the at least one of the one or more optical components 420 can determine reflectance, transmittance, fluorescence, luminescence, and/or polarization state of the incubated droplet 166. For example, the one or more optical components 420 can detect at least an optical property of droplets that include a chemiluminescent assay.

In some embodiments, the one or more optical components 420 may be on-chip embedded. In some embodiments, the one or more optical components 420 may include a detection diode disposed via a thin film fabrication. In some embodiments, the diode may include a thin film filter deposited on the diode. In some embodiments, the one or more optical components 420 may include a component for excitation illumination. In some embodiments, the one or more optical components 420 may include fluorescent or other optics for characterizing the content of droplets before dispensing of droplets for downstream processing.

In some embodiments, a microfluidic system may include both the electrical detection system described with respect to FIG. 3 and the optical components described with respect to FIG. 4.

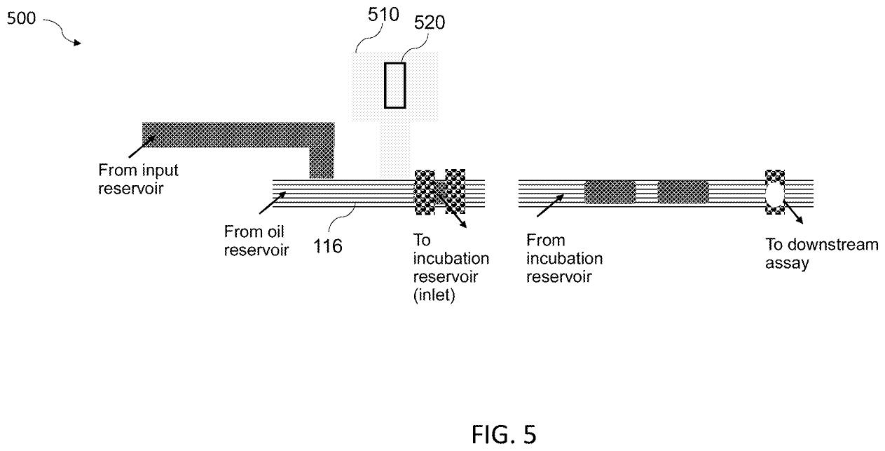

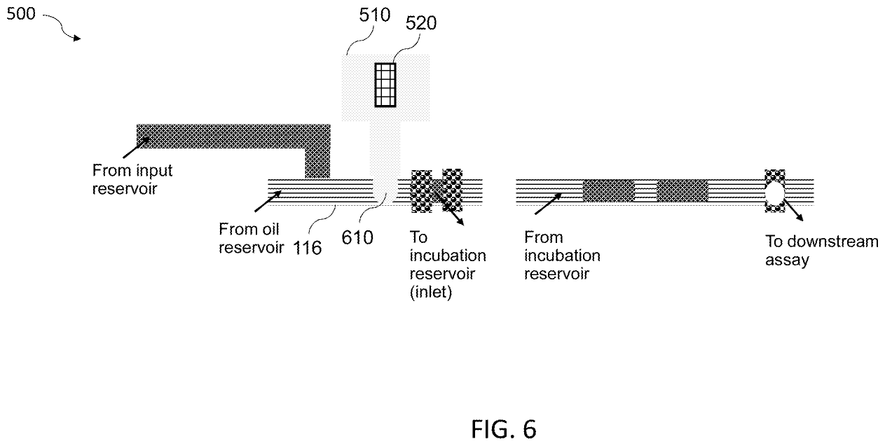

FIG. 5 and FIG. 6 shows top views of examples of microfluidic systems 500 according to various embodiments. The microfluidic system 500 may include a valving structure 510. The valving structure 510 may include a resistor 520 and a valving portion 610. The valving structure 510 may be or include a micro valve, a thermopneumatic valve, or a thermopneumatic microfluidic valve. The valving structure 510 may be disposed at the upstream side of the inlet 112a. The valving structure 510 can prevent backflow from the incubation reservoir 112.

The microfluidic system 500 in FIG. 5 shows an open state where the valving portion 610 does not block the first fluidic network 116. The microfluidic system 500 in FIG. 6 shows a close state where the valving portion 610 blocks a portion of the first fluidic network 116 and prevents backflow from the incubation reservoir 112.

In some embodiments, the valving structure 510 may include the resistor 520. When the resistor 520 is on, a gas or air in a chamber of the valving structure 510 can expand into the first fluidic network 116 and block a portion of the first fluidic network 116, thereby preventing backflow from the incubation reservoir 112. In this case, such a gas or air can act as the valving portion 610.

In some embodiments, the valving structure 510 may include a main channel, a passage that includes an opening in fluid communication with the main channel (e.g., the first fluidic network 116), and a side chamber to house a volume of trapped gas. The side chamber is communicably attached to the passage to control flow along the main channel. The side chamber is to be larger in volume than the main channel to which the trapped gas expands. The valving structure 510 may include at least the following two states at a given time: 1) an open state in which the main channel is open and flow proceeds through the main channel, or 2) a closed state in which the trapped gas within the side chamber is to expand within the passage and block the flow in the main channel (e.g., backflow from the incubation reservoir 112).

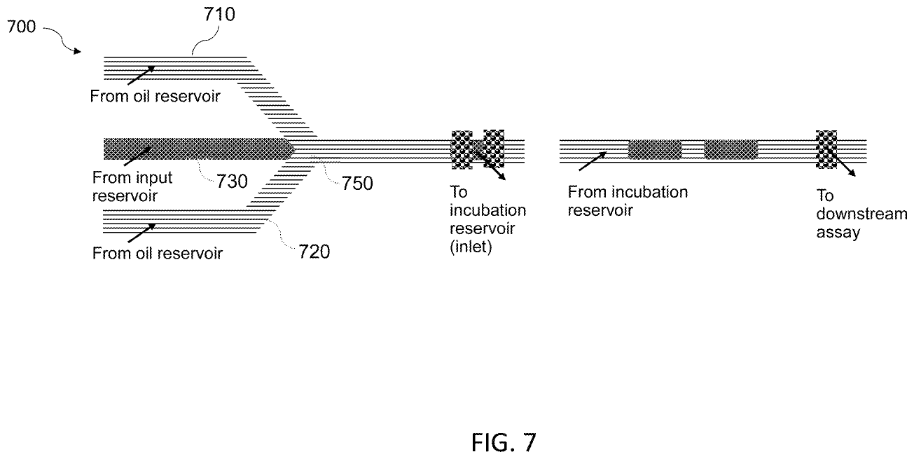

FIG. 7 shows top views of examples of microfluidic systems 700 according to various embodiments. The microfluidic system 700 may include a first channel 710, a second channel 720, and a reagent channel 730 that collectively form the first fluidic network 116. The first channel 710 and the second channel 720 can be fed by the immiscible fluid reservoir 108 (or by respective immiscible fluid reservoir). The immiscible fluid 158 then can flow via a junction 750 through the inlet 112a. The reagent channel 730 can be fed by the reagent reservoir 104, and the reagent 154 then can flow via the junction 750. Since the junction 750 can act as a droplet generator encapsulating the reagent 154 as discussed above, the droplet 162 can be generated at the junction 750 and flow through the inlet 112a, thereby feeding into the incubation reservoir 112 (e.g., by the fluid actuator, inverted TIJ nozzle, etc.). Although the microfluidic system 700 is depicted to include two channels, in some embodiments, the microfluidic system 700 may include any number of multiple channels of droplet generation feeding into the incubation reservoir 112.

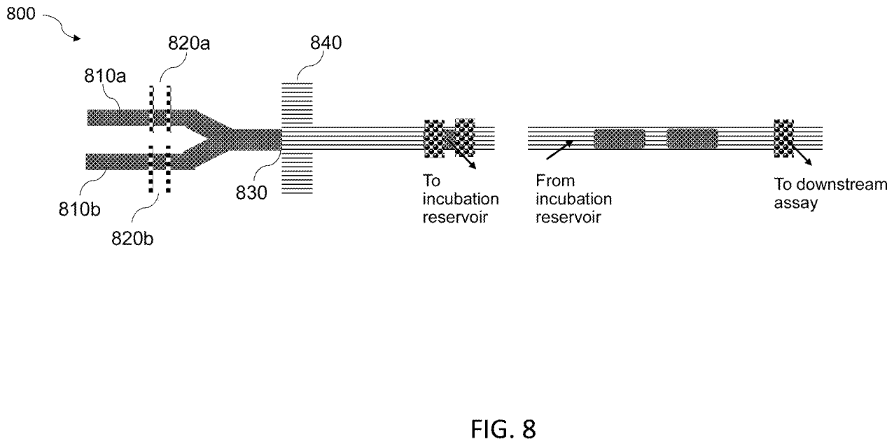

FIG. 8 shows top views of examples of microfluidic systems 800 according to various embodiments. The microfluidic system 800 may include a first reagent channel 810a, a second reagent channel 810b, first impedance sensing electrodes 820a, second impedance sensing electrodes 820b, and an immiscible fluid channel 840. Although depicted to include two reagent channels, in some embodiments, the microfluidic system 800 may include one or more reagent channels. In some embodiments, a plurality of reagent channels of the microfluidic system 800 may each include a droplet generation geometry.

The first impedance sensing electrodes 820a may detect a first content (e.g., cells or other particles) in the reagent of the first reagent channel 810a, and the second impedance sensing electrodes 820b may detect a second content in the reagent of the second reagent channel 810b. Although the impedance sensing electrodes are shown, the microfluidic system 800 may include the optical components described with respect to FIG. 4.

In some embodiments, each of the first reagent channel 810a and the second reagent channel 810b can generate droplets with the respective reagent. In some embodiments, the reagents from the first reagent channel 810a and the second reagent channel 810b may flow into the junction 830, which then generates droplets encapsulated by the immiscible fluid fed by the immiscible fluid channel 840. In some embodiments, the microfluidic system 800 may be used to merge different reagents from a plurality of reagent channels (e.g., the first reagent channel 810a, the second reagent channel 810b). For example, a first cell from the first reagent channel 810a and a second cell from the second reagent channel 810b can merge. In some embodiments, the first impedance sensing electrodes 820a and the second impedance sensing electrodes 820b can register merging of cells with beads.

In certain embodiments, multiple channels of droplet generation may go into a single incubation chamber. Separator droplets may be used to separate different droplet batches, so that some structure remains when the droplets are retrieved from the incubation reservoir.

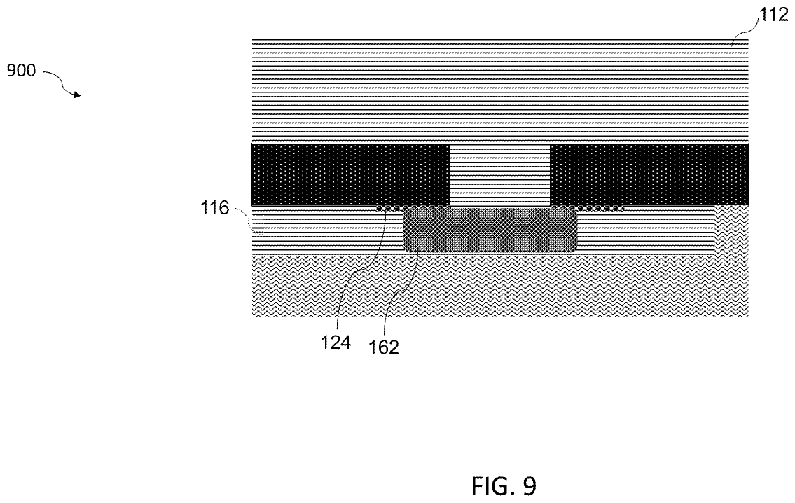

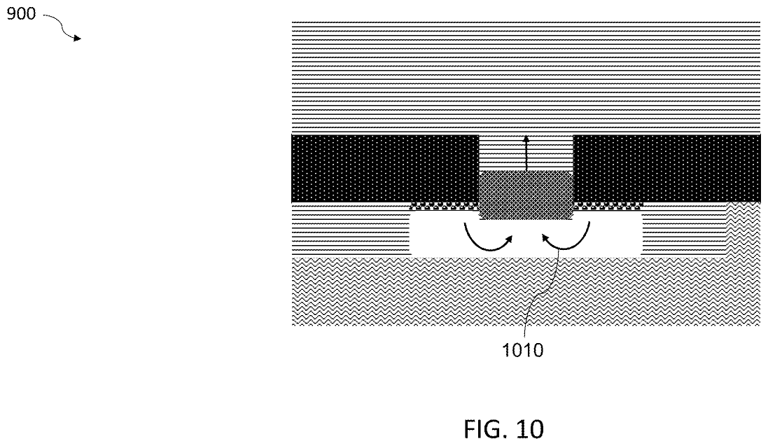

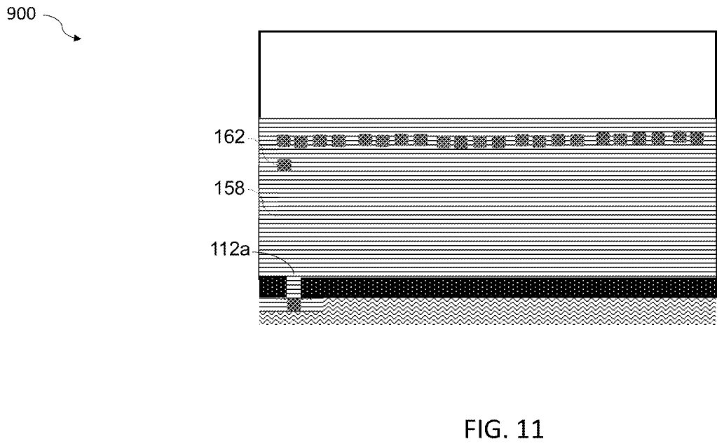

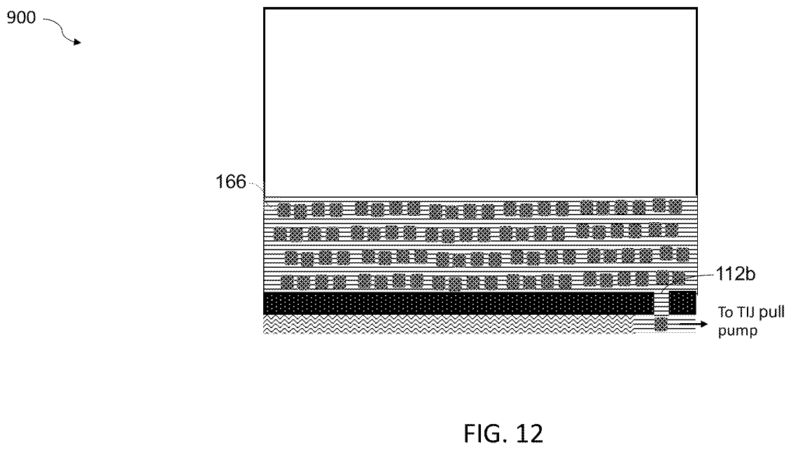

FIG. 9 to FIG. 12 show cross-sectional views of examples of microfluidic systems 900 according to various embodiments. More specifically, FIG. 9 to FIG. 12 show a detailed view of the incubation reservoir 112. Referring to FIG. 9, a detailed view of the microfluidic system 900 shows the incubation reservoir 112, the first fluidic network 116, the first fluid actuator 124, and the droplet 162. During various operations of the microfluidic system 900, the droplet may be positioned around the first fluid actuator 124. Referring to FIG. 10, the first fluid actuator 124 can generate an expanding bubble, whose momentum 1010 pushes the droplet 162 upward, thereby introducing the droplet 162 into the incubation reservoir 112. Referring to FIG. 11, a detailed view of the microfluidic system 900 shows the incubation reservoir 112 including a plurality of droplets 162. As shown in FIG. 11, the incubation reservoir 112 may be filled or partially filled with the immiscible fluid 158, and the droplet 162 may float to the top of the immiscible fluid 158 (e.g., a surface of the immiscible fluid 158 in the incubation reservoir 112). In some embodiments, the immiscible fluid 158 may be denser than the droplet 162 (e.g., FC40, NOVEC-HFE7500, NOVEC-HFE7100, NOVEC-HFE7×00) so that the droplet 162 can float up to the top of the incubation reservoir 112 thereby preventing the droplet 162 from loitering near the entrance of the incubation reservoir 112 (e.g., the inlet 112a). This can prevent the droplet 162 from coalescing with other droplets. Referring to FIG. 12, a detailed view of the microfluidic system 900 shows the incubation reservoir 112 including a plurality of incubated droplets 166. Once the incubation is complete, the second fluidic network 128 may pull the incubated droplet 166 and/or the immiscible fluid 158 out of the incubation reservoir 112 through the outlet 112b, or otherwise the incubated droplet 166 and/or the immiscible fluid 158 may be introduced into the second fluidic network 128. The incubated droplet 166 introduced into the second fluidic network 128 then can flow through the second fluidic actuator 132.

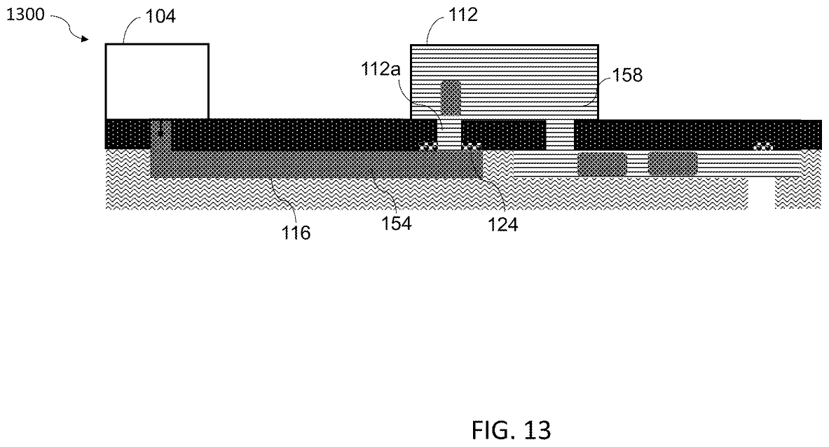

FIG. 13 shows cross-sectional views of examples of microfluidic systems 1300 according to various embodiments. In some embodiments, an immiscible fluid reservoir may be omitted. In such a case, the incubation reservoir 112 may be pre-filled (or partially filled) with the immiscible fluid 158, or otherwise supplied the immiscible fluid 158. The reagent 154 fed into the first fluidic network 116 can flow through the first fluidic actuator 124 (e.g., without forming a droplet). The first fluid actuator 124 may act as a valve to the incubation reservoir 112, while generating a droplet of the reagent 154. For example, the first fluid actuator 124 can generate a droplet of the reagent 154, and introduce the droplet into the incubation reservoir 112.

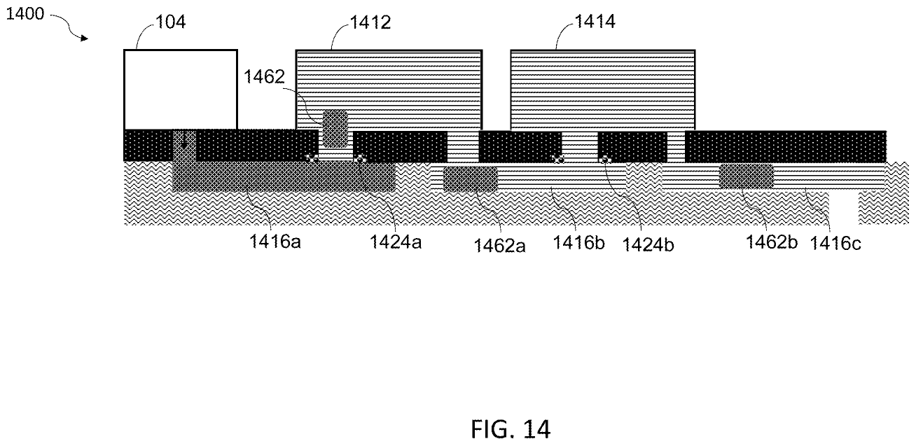

FIG. 14 shows cross-sectional views of examples of microfluidic systems 1400 according to various embodiments. As can be seen in FIG. 14, as opposed to the microfluidic system 1300, the microfluidic system 1400 may alternatively include a first incubation reservoir 1412 and a second incubation reservoir 1414. In some embodiments, the microfluidic system 1400 may include a plurality of incubation reservoirs. The microfluidic system 1400 may further include a first fluidic network 1416a, a second fluidic network 1416b, a third fluidic network 1416c, a first fluid actuator 1424a, and a second fluid actuator 1424b. The microfluidic system 1400 may include a droplet 1462, a first incubated droplet 1462a, and a second incubated droplet 1462b.

In some embodiments, the reagent reservoir 1404 can fed reagents into the first fluidic network 1416a. The reagents can flow through the first fluid actuator 1424a, and can be introduced into the first incubation reservoir 1412, as the droplet 1462 by the first actuator 1424a. The first incubation reservoir 1412 then can incubate the droplet 1462 at a first incubation condition. Once the first incubation is complete in the first incubation reservoir 1412, the first incubated droplet 1462a can be introduced into the second fluidic network 1416b. The first incubated droplet 1462a can be introduced into the second incubation reservoir 1414, by the second fluid actuator 1324b for second incubation in the second incubation reservoir 1414. Once the second incubation is complete, the second incubated droplet 1462b can be introduced into the third fluidic network 1416c, from which the second incubated droplet 1462b can be introduced/ejected for further processing and/or downstream analysis.

In some embodiments, a plurality of incubation reservoirs may perform different incubation processes on droplets. For example, the first incubation reservoir 1412 may be for incubation at a first condition, and the second incubation reservoir 1414 may be for incubation at a second condition. For example, the first incubation reservoir 1412a and/or the second incubation reservoir 1412b may incubate only some droplets that contain a specific content.

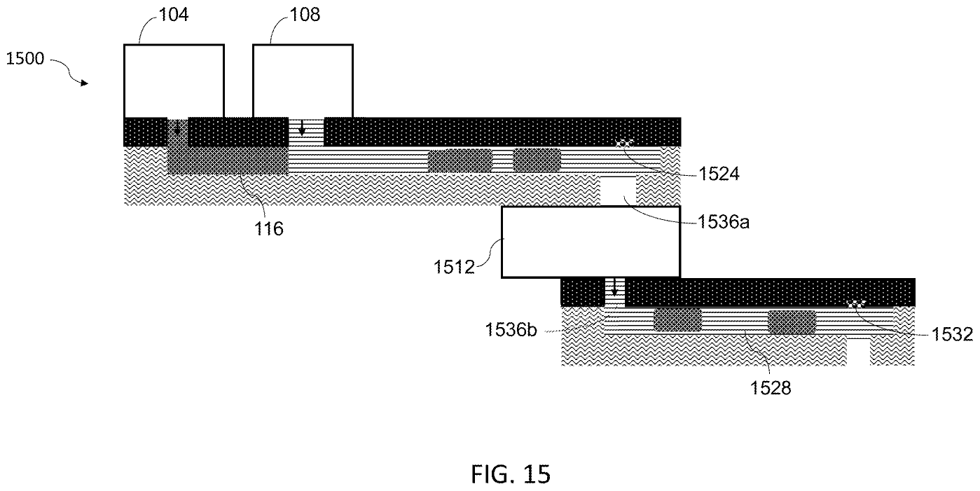

FIG. 15 shows cross-sectional views of examples of microfluidic systems 1500 according to various embodiments. Various components described herein may be arranged in various ways. In some embodiments, the microfluidic system 1500 may include non-coplanar reservoirs. In some embodiments, as shown in FIG. 15, an incubation reservoir 1512 may be disposed below the first fluidic network 116. An output 1536a disposed at the upstream side of the incubation reservoir 1512 may be connected to an inlet portion of the incubation reservoir 1512 such that droplets can be introduced into the incubation reservoir 1512. In some embodiments, the flow of the droplets into the incubation reservoir 1512 may be controlled by a first fluid actuator 1524. For example, the first fluid actuator 1524 may act as a valve so as to control the flow of the droplets into the incubation reservoir 1512. Once the incubation is complete in the incubation reservoir 1512, the incubated droplets can be introduced into a second fluidic network 1528 through an input 1536b of the second fluidic network 1528. For example, a second fluid actuator 1532 may act as a pull pump, or otherwise the incubation reservoir 1512 can introduce the incubated droplets into the second fluidic network 1528. The incubated droplets introduced to the second fluidic network 1528 can be ejected by the second fluid actuator 1532 for downstream assays or further processing.

In some embodiments, the microfluidic system 1500 may be used, when for example, the immiscible fluid is less dense. For example, the immiscible fluid is less dense than the reagents or droplets. In this case, the droplet can sink to the bottom of the incubation reservoir 1512. Example immiscible fluid may include silicon oil (e.g., 3 cSt, 5 cSt), mineral oil, kerosene, Isopar-G, Isopar-L. This configuration can prevent droplets from loitering near the entrance (e.g., the output 1536a) of the incubation reservoir 1512. Droplet loitering near the entrance can cause coalescence with other droplets, and this can be avoided.

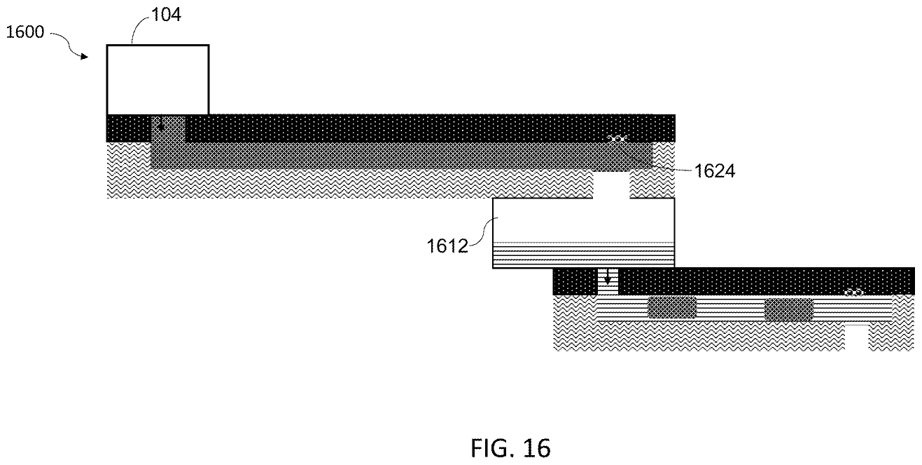

FIG. 16 shows cross-sectional views of examples of microfluidic systems 1600 according to various embodiments. In some embodiments, as opposed to the microfluidic system 1500 shown in FIG. 15, an immiscible fluid reservoir may be omitted in the microfluidic system 1600. In this case, a first fluid actuator 1624 may act as a droplet generator to generate droplets and introduce the droplets into an incubation reservoir 1612. The incubation reservoir 1612 may be pre-filled (or partially filled, or otherwise supplied) with immiscible fluid, which then can encapsulate the introduced droplet. After the introduced droplet is incubated in the incubation reservoir 1612, the incubated droplet can be introduced into the second fluidic network.

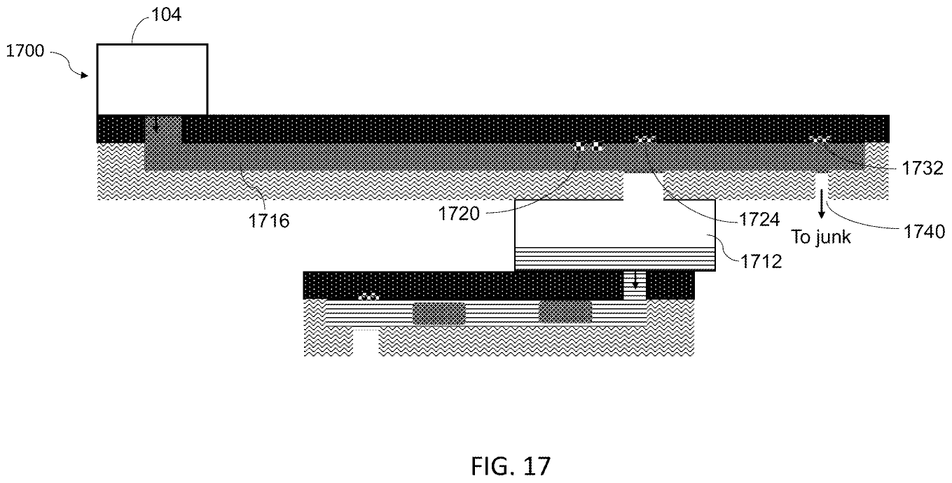

FIG. 17 shows cross-sectional views of examples of microfluidic systems 1700 according to various embodiments. In some embodiments, as opposed to the microfluidic system 1600, a first fluidic network 1716 of the microfluidic system 1700 may include impedance sensing electrodes 1720, a first fluid actuator 1724, a second fluid actuator 1732, and a junk output 1740. The impedance sensing electrodes 1720 can detect a certain content in reagents fed by the reagent reservoir 104 or droplets in the first fluidic network 1716. Based on a detection of the certain content in the reagents or the droplets in the first fluidic network 1716, the first fluid actuator 1724 can introduce (e.g., eject) the reagent or the droplets into the incubation reservoir 1712. For example, the first fluid actuator 1724 can generate a droplet while acting as a valve, provide the reagent as a droplet to the incubation reservoir 1712 where the droplet is encapsulated by oil or immiscible fluid. In some embodiments, when the first fluid actuator 1724 does not eject (or otherwise introduce) the reagent or droplet into the incubation reservoir 1712, the reagent can flow through the junk output 1740. The second fluid actuator 1732 then can eject the reagent adjacent thereto through the junk output 1740. In some embodiments, if a cell is detected, the volume of fluid including the cell can be jetted into the incubation reservoir 1712. Otherwise the fluid is jetted to junk. This way majority of the droplets contain a cell, and the incubation reservoir volume can be used more efficiently. In some embodiments, the microfluidic system 1700 may be used to filter a certain content. For example, the impedance sensing electrodes 1720 can detect a certain content not to be introduced into the incubation reservoir 1712. When the impedance sensing electrodes 1720 detect such a content, the first fluid actuator 1724 does not eject the reagent or droplet including that content, which then flows through the second fluid actuator 1732 and can be ejected through the junk output 1740.

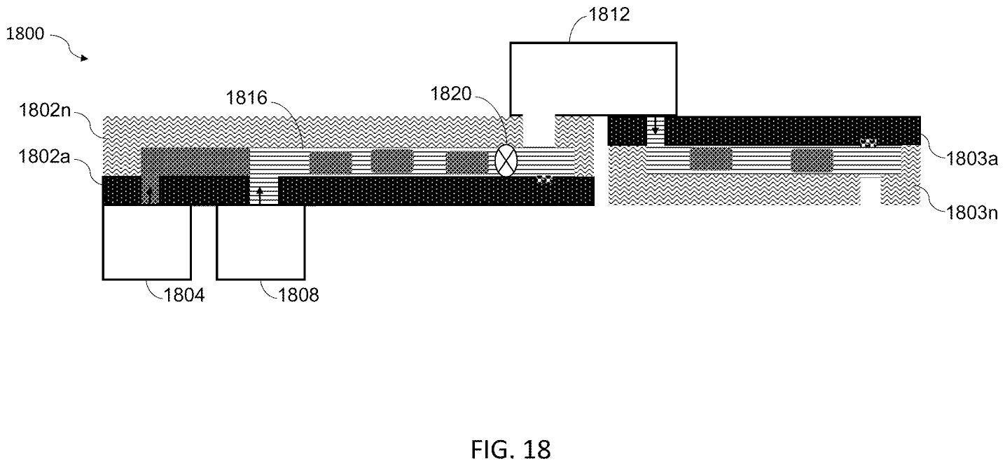

FIG. 18 shows cross-sectional views of examples of microfluidic systems 1800 according to various embodiments. The microfluidic system 1800 may include a first network layer 1802n, a first active layer 1802a, a second network layer 1803n, a second active layer 1803a, a reagent reservoir 1804, an immiscible fluid reservoir 1808, a first fluidic network 1816, a valve 1820, and an incubation reservoir 1812. In some embodiments, the microfluidic system 1800 may include non-coplanar reservoirs. In some embodiments, arrangement of the first active layer 1802a and the first network layer 1802n may be different from that of the second active layer 1803a and the second network layer 1803n. For example, the first active layer 1802a and the first network layer 1802n may be inverted with respect to the second active layer 1803a and the second network layer 1803n. For example, the first active layer 1802a may be disposed below the first network layer 1802n while the second active layer 1803a may be disposed at the top of the second network layer 1803n, or vice versa. In some embodiments, the reagent reservoir 1804 and/or the immiscible fluid reservoir 1808 may be disposed below the first fluidic network 1816, for example when the first active layer 1802a is disposed at the bottom of the first network layer 1802n.

In some embodiments, the microfluidic system 1800 may include the valve 1820 disposed at the upstream side of the incubation reservoir 1812 in the first fluidic network 1816. The valve 1820 can prevent backflow from the incubation reservoir 1812.

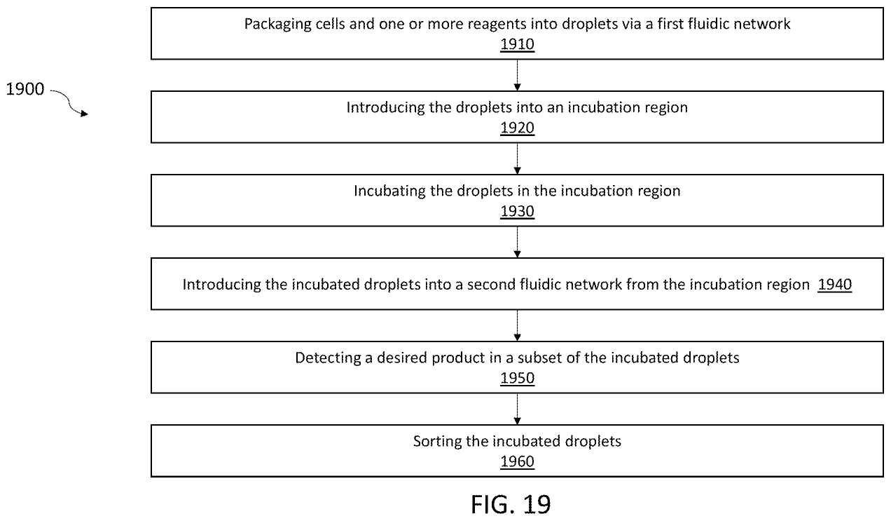

FIG. 19 shows an example flowchart of a method 1900 according to various embodiments. The method 1900 may be to select cells producing a specific product using a microfluidic device. The method 1900 may be performed using any of the microfluidic systems described herein or any components thereof. The method 1900 may include packaging, into droplets, via a first fluidic network of the microfluidic device, cells that are in a cell suspension, wherein the cells are packaged into the droplets such that each droplet includes (1) no more than one cell from the cell suspension, and (2) one or more reagents from one or more reagent reservoirs of the microfluidic device. The method 1900 may include ejecting (or otherwise introducing) droplets into an incubation region of the microfluidic device. The method 1900 may include incubating the droplets in the incubation region at a temperature and for a period of time. The method 1900 may include pulling (or otherwise introducing) incubated droplets from the incubation region into a second fluidic network of the microfluidic device. The method 1900 may include detecting a desired product in a subset of the incubated droplets pulled from the incubation region. The method 1900 may include sorting the incubated droplets based on whether the desired product is detected in the incubated droplets.

In some embodiments, the method 1900 may include, packaging cells and one or more reagents into droplets via a first fluidic network, introducing the droplets into an incubation region, incubating the droplets in the incubation region, introducing the incubated droplets into a second fluidic network from the incubation region, detecting a desired product in a subset of the incubated droplets, and sorting the incubated droplets.

For example, as shown in FIG. 19, the method 1900 may begin by packaging cells and one or more reagents into droplets via a first fluidic network (block 1910). In some embodiments, the method 1900 may include using a measurement system to detect the cells prior to packaging the cells into the droplets, the measurement system including a pair of impedance electrodes or one or more optical components. In some embodiments, the method 1900 may include assaying the desired product in the reagents or cells. In some embodiments, the method 1900 may include packaging the cells into the droplets using oil or immiscible fluid from an oil reservoir (or immiscible fluid reservoir) of the microfluidic device. In response to generation of the droplets and/or the droplets having moved to an actuator region, the method 1900 may proceed to introducing the droplets into an incubation region (block 1920), for example, by a fluid actuator. In response to introduction of the droplets into the incubation region, the method 1900 may proceed to incubating the droplets in the incubation region (block 1930). In response to completion of the incubation, the method 1900 may proceed to introducing the incubated droplets into a second fluidic network from the incubation region (block 1940). In some embodiments, the method 1900 may include detecting a desired product in a subset of the incubated droplets (block 1950) in response to the incubated droplets having moved to the second fluid network. The method 1900 may end by sorting the incubated droplets (block 1960) in response to a detection of the desired product and/or the incubated droplets having moved to the second fluid network. In some embodiments, the method 1900 may include sorting the incubated droplets using impedance electrodes or one or more optical components, wherein the one or more optical components may include an excitation illumination and detect collection wavelengths. For example, the method 1900 may include detect or measure an electrical property of the reagents, cells or droplets using the impedance electrodes, and in response to a detection or measurement, the method 1900 can proceed to determining whether to incubate, introduce for downstream assays, or waste the reagents, cells, or droplets. For example, the method 1900 may include detect or measure an optical property of the reagents, cells or droplets using the one or more optical components, and in response to a detection or measurement, the method 1900 can proceed to determining whether to incubate, introduce for downstream assays, or waste the reagents, cells, or droplets.

The microfluidic system and the methods thereof described with respect to FIG. 1 to FIG. 19 may be used to generate protein libraries. For example, the microfluidic system may dispense the resulting library, one protein variant at a time, into a multiwell plate for downstream analysis. The microfluidic systems may include an input reservoir (or reagent reservoir), an oil reservoir (or immiscible fluid reservoir), and a channel (or fluidic networks) with an incubation region (or incubation reservoir). Downstream of the incubation region may include magnetic actuation regions, and/or side channels with resistors (e.g., TIJ resistors) to move waste product there. The channel or a junction formed therein may form a droplet generator, transport the droplets downstream, and perform magnetic operations on the train of droplets. The input reservoir may contain nucleic acid templates, magnetic beads with affinity to target protein of fragments thereof, and reagents for cell-free translation (and/or transcription) reaction. The nucleic acid template may be at a low concentration (limiting dilution concentration) such that about 37% of the droplets contain 1 template, and less than 5% of the droplets contain two or more templates. Each template may encode for a different variant of the protein. The microfluidic systems may combine the template with the cell-free translation (and/or transcription) reagents to produce protein, purify the protein using magnetic beads and ink jet actuator (e.g., TIJ) driven droplet manipulation, elute the protein, and eject it into a multiwell plate.

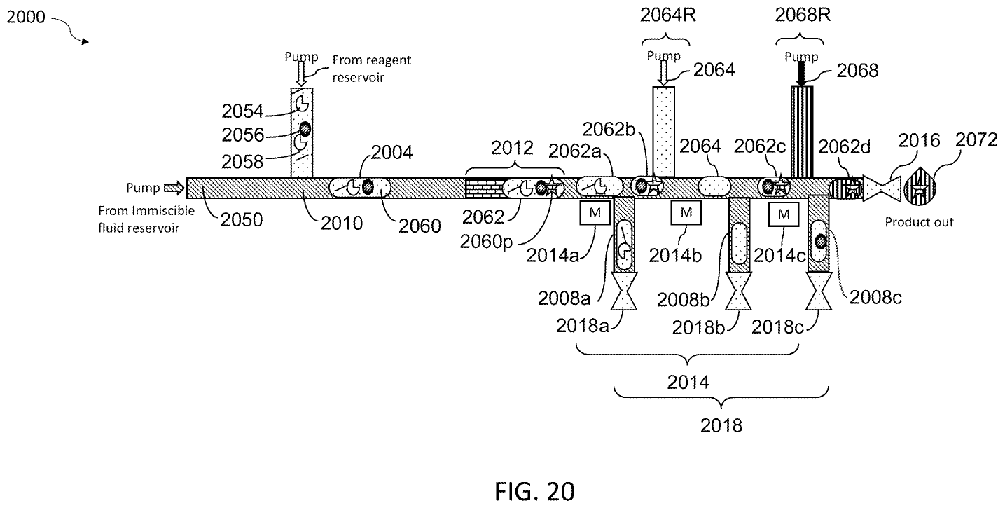

FIG. 20 shows top views of examples of microfluidic systems 2000 according to various embodiments. The microfluidic system 2000 may include an input reservoir (or reagent reservoir) providing NA templates 2054, magnetic beads 2056, and cell-free translation (and/or transcription) reagents 2058. The microfluidic system 2000 may include an immiscible fluid reservoir (or oil reservoir) providing an oil 2050 (or immiscible fluid). The microfluidic system 2000 may include a channel 2004 (sometimes referred to as a fluidic network), a junction 2010 to generate a droplet 2060, an incubation region 2012 where protein of interest 2060p is incubated, a magnetic actuation region 2014 including a first magnet 2014a disposed at the upstream side of a first side channel 2008a, a second magnet 2014b disposed at the upstream side of a second side channel 2008b, and a third magnet 2014c disposed at the upstream side of a third side channel 2008c. The microfluidic system 2000 may include a separation region 2018, including one or more side channels 2008a-c. The side channels 2008a-c may include a respective valve 2018a-c. The microfluidic system 2000 may include a washing region providing a washing reagent 2064 and an elution region providing an elution reagent 2068. The microfluidic system 2000 may include an output valve 2016 to control ejection of a product 2072. In some embodiments, the microfluidic system 2000 may operate with external drivers.

The microfluidic system 2000 may include a channel. The microfluidic system 2000 may include a set of reservoirs feeding the channel, the set of reservoirs including an input reservoir including magnetic beads and an oil reservoir. In embodiments, the microfluidic system 2000 may include a droplet generator downstream of a junction between the channel and the input reservoir. In other embodiments, the junction of the channel and the input reservoir (e.g., the point at which the channel and the input reservoir connect or otherwise meet) may function as a droplet generator. The microfluidic system 2000 may include an incubation region downstream of the junction. The microfluidic system 2000 may include, downstream of the incubation region, one or more magnetic actuation regions, and one or more side channels.

In various embodiments, the oil 2050 and the droplet 2060 may be substantially identical to the immiscible fluid and the droplet disclosed with respect to FIG. 1 to FIG. 19. The channel 2004 may be substantially identical to the first fluidic network and/or the second fluidic network disclosed with respect to FIG. 1 to FIG. 19. The junction 2010 may be substantially identical to the junction disclosed with respect to FIG. 1 to FIG. 19. The output valve 2016 substantially identical to the valving structure and/or the valve disclosed with respect to FIG. 1 to FIG. 19.

The input reservoir (or the reagent reservoir) may include nucleic acid (NA) template 2054, and can provide the NA template 2054. The NA template 2054 may be at a low concentration (limiting dilution concentration) such that at least about 37% of the droplets contain 1 template, and less than 5% of the droplets contain two or more templates. Each of the NA template 2054 may encode for a different variant of the protein or fragment thereof. The microfluidic system 2000 may combine the NA template 2054 with the cell-free translation (and/or transcription) reagents 2058 to produce protein (e.g., the protein of interest 2060p). The microfluidic system 2000 may purify the protein using the functionalized magnetic beads 2056 and ink jet driven (e.g., TIJ) droplet manipulation. The microfluidic system 2000 may elute the protein, and eject it into a multiwell plate. In some embodiments, the NA template 2054 may be with primers. For example, the NA template 2054 may be or include mRNA or DNA encoding a target protein or fragment thereof. In some embodiments, the NA template 2054 may be linear or a circular (e.g., plasmid). In some embodiments, the NA template 2054 may include a region that encodes for a tag in protein (e.g. His Tag, or FLAG tag) so that it can bind to an affinity probe on the magnetic beads 2056.

The NA template 2054 may be a DNA template that can be included in a vector, that are operatively linked to regulatory elements such as for example, promoters linked to the NA template. In some embodiments, any vector containing T7, SP6 or T3 promoters may be used (e.g., as long as the mixture contains phage-derived RNA polymerase (T7, T3 or SP6)). For a eukaryotic derived system, ATG start codon in the sequence may be the first ATG encountered following the transcription start site. In some embodiments, this ATG may be included in the Kozak consensus sequence. In some embodiments, stop codon should be included at the 3′-terminus of the sequence. In some embodiments, a synthetic poly(A) tail may be included following the stop codon. Example template design includes:

SP6 5′(N)₆₋₁₀-TATTTAGGTGACACTATAG(N)₃₋₆-CCACCATGG-(N)₁₇₋₂₂-3′ (SEQ ID NO: 1), where TATTTAGGTGACACTATAG (SEQ ID NO: 2) is SP6 Promoter, CCACCATGG (SEQ ID NO: 3) is Kozak region, and (N)₁₇₋₂₂ is Sequence-specific Nucleotides.

T7 5′(N)₆₋₁₀-TAATACGACTCACTATAGGG(N)₃₋₆-CCACCATGG-(N)₁₇₋₂₂-3′ (SEQ ID NO: 4), where TAATACGACTCACTATAGGG (SEQ ID NO: 5) is T7 Promoter, CCACCATGG (SEQ ID NO: 7) is Kozak region, and (N)₁₇₋₂₂ is Sequence-specific Nucleotides.

In some embodiments, sequence may be inserted into a commercially available template.

The input reservoir (or the reagent reservoir) may include the magnetic beads 2056, and can provide the magnetic beads to the channel 2004. The magnetic beads 2056 may be with affinity to target protein. In some embodiments, the magnetic beads 2056 may be coated an affinity binding reagent (e.g. Antibody) against a tag, such as a FLAG tag on the protein.

The input reservoir (or the reagent reservoir) may include the cell-free translation (and/or transcription) reagents 2058, and can provide the cell-free translation (and/or transcription) reagents 2058. In some embodiments, the cell-free translation (and/or transcription) reagents 2058 may be a cell-free extraction reagent mixture broadly available (e.g., Thermo Fisher). In some embodiments, the cell-free translation (and/or transcription) reagents 2058 or the mixture may include lysed E. Coli. or Wheat Germ. For example, the cell-free translation (and/or transcription) reagents 2058 or the mixture may include RNA polymerases for mRNA transcription (if the template is DNA), ribosomes for polypeptide translation (and/or transcription), tRNA and amino acids, enzymatic cofactors (e.g. Calcium), an energy source (e.g. adenosine triphosphate), or cellular components essential to protein folding and post-translational (and/or post-transcriptional) modifications (from the cell lysate). For example, the cell-free translation (and/or transcription) reagents 2058 or the mixture may be a commercially available product.

In some embodiments, for example when starting with mRNA template, the translation (and/or transcription) systems (e.g., the cell-free expression systems, the cell-free translation (and/or transcription) reagents 2058) may include ribosomes, tRNAs, aminoacyl-tRNA synthetases, initiation, elongation and termination factors, amino acids, energy sources (ATP, GTP), or energy regenerating systems and salts (Mg2⁺, K⁺, etc.). In some embodiments, the translation (and/or transcription) system or the cell-free translation (and/or transcription) reagents 2058 may include creatine phosphate and creatine phosphokinase serving as energy regenerating system (eukaryotic derived) or phosphoenol pyruvate and pyruvate kinase (prokaryotic derived). In some embodiments, for example when starting with DNA template, the translation (and/or transcription) systems (e.g., coupled translation (and/or transcription) and transcription systems, the cell-free translation (and/or transcription) reagents 2058) may include ribosomes, tRNAs, aminoacyl-tRNA synthetases, initiation, elongation and termination factors, amino acids, energy sources (ATP, GTP), energy regenerating systems and salts (Mg2⁺, K⁺, etc.), or phage-derived RNA polymerase (T7, T3 or SP6) allowing the expression of genes cloned downstream of a T7, T3 or SP6 promoter. The translation (and/or transcription) systems may include creatine phosphate and creatine phosphokinase serving as energy regenerating system (eukaryotic derived) or phosphoenol pyruvate and pyruvate kinase (prokaryotic derived).

The incubation region 2012 may be substantially identical to the incubation reservoir descried with respect to FIG. 1 to FIG. 19. For example, the incubation region 2012 may be or include one or more incubation chambers or one or more incubation reservoirs described with respect to FIG. 1 to FIG. 19. The incubation region 2012 may generate, incubate, or otherwise introduce the protein of interest 2060p into the droplet 2060, thereby producing protein libraries. For example, the protein of interest 2060p may be or include antibodies with strong and specific binding to an antigen for therapy, lipase enzymes in laundry detergent that are superior at breaking down grease stains while surviving typical laundering and storage conditions and not breaking down fabric and dye, etc.

The microfluidic system 2000 may include the magnetic actuation region 2014. In some embodiments, the magnetic actuation region 2014 may be disposed downstream of the incubation region 2012. The magnetic actuation region 2014 may include the first magnet 2014a, the second magnet 2014b, and the third magnet 2014c. In some embodiments, each of the first magnet 2014a, the second magnet 2014b, and the third magnet 2014c may form a magnetic actuation region. In some embodiments, the first magnet 2014a may be disposed at the upstream side of the first side channel 2008a, the second magnet 2014b may be disposed at the upstream side of the second side channel 2008b, and the third magnet 2014c may be disposed at the upstream side of the third side channel 2008c. In some embodiments, any of the magnets 2014 a-c may be or include a permanent magnet.

The microfluidic system 2000 may include the separation region 2018. The separation region 2018 may include the side channels 2008a-c connected to the channel 2004. Each of the side channels 2008a-c may include the respective valve 2018a-c. In some embodiments, at least one of the side channels 2008 may be formed adjacent to at least one of the magnets 2014a-c. In some embodiments, the side channels 2008a-c may include a respective valve 2018a-c. The separation region 2018 may be disposed downstream of the one or more magnetic actuation regions, wherein the one or more side channels are part of the separation region.

The microfluidic system 2000 may include a washing region 2064R providing a washing reagent 2064. The microfluidic system 2000 may include a washing reagent reservoir to provide the washing reagent 2064, for example downstream of at least one of the magnetic actuation region 2014 or the incubation region 2012. The washing reagent 2064 and/or the washing region 2064R are to mix with at least a portion of incubated droplets and dilute the contents as to wash them. In some embodiments, the microfluidic system 2000 may include several washing regions. For example, such washing regions may be placed in series to achieve a sufficiently pure product.

The microfluidic system 2000 may include an elution region 2068R providing an elution reagent 2068. The microfluidic system 2000 may include an elution reagent reservoir to provide the elution reagent 2068, for example downstream of at least one of the magnetic actuation region 2014 or the incubation region 2012. The elution reagent 2068 and/or the elution region 2068R are to mix with at least a portion of incubated droplets, thereby separating protein from the magnetic beads. In some embodiments, the elution reagent 2068 and/or the elution region 2068R may be to release the coupling of a tag on the protein from the affinity partner on the bead (e.g. 200 mM imidazole solution for His Tag). In some embodiments, the elution reagent 2068 and/or the elution region 2068R may elute the protein from the bead (e.g. with imidazole solution for the His-tag).

Still referring to FIG. 20, the immiscible fluid reservoir can provide oil 2050 to the channel 2004. The oil 2050 may flow through the channel 2004 and/or the side channels 2008. The reagent reservoir (or input reservoir) can provide the NA template 2054, the magnetic beads 2056, and the cell-free translation (and/or transcription) reagents 2058 to the channel. In some embodiments, the reagent reservoir (or input reservoir) can introduce the NA template 2054, the magnetic beads 2056, and the cell-free translation (and/or transcription) reagents 2058 into the junction 2010, which then can generate the droplet 2060. The droplet 2060 may include the NA template 2054, the magnetic beads 2056, and the cell-free translation (and/or transcription) reagents 2058. When the droplet 2060 enters the incubation region 2012, the incubation region 2012 can incubate the droplet 2060, or otherwise generate the protein of interest 2060p in the droplet 2060, thereby generating the incubated droplet 2062.

As a downstream process of the incubation, the incubated droplet 2062 may enter the magnetic actuation region 2014 and/or the separation region 2018. Although depicted to include the three magnets 2014a-c, the magnetic actuation region 2014 may include any number of magnets. When the incubated droplet 2062 passes through the first magnet 2014a, or otherwise when interacting with the first magnet 2014a, the first magnet 2014a may retract any part of the incubated droplet 2062. For example, the first magnet 2014a may retract a portion of the cell-free translation (and/or transcription) reagents (referred to as a first retracted portion 2062a) from the incubated droplet 2062. The first side channel 2008a and/or the valve 2018a may then pull the first retracted portion 2062a to waste. The remaining portion (referred to as, a first processed droplet 2062b) may then enter the washing region 2064R. A washing reagent reservoir (not depicted) can provide the washing reagent 2064 to the channel 2004 and wash the first processed droplet 2062b. When the first processed droplet 2062b passes through the second magnet 2014b, or otherwise when interacting with the second magnet 2014b, the second magnet 2014b may separate the washing reagent 2064 from the first processed droplet 2062b. The second side channel 2008b and/or the valve 2018b may then pull the washing reagent 2064 to waste. The remaining portion (referred to as, a second processed droplet 2062c) may then travel toward the elution region 2068R. An elution reagent reservoir (not depicted) can provide the elution reagent 2068 to the channel 2004. Then the elution reagent 2068 can separate the magnetic beads 2056 from the protein of interest 2060p in the second processed droplet 2062c. When the second processed droplet 2062c that has been eluted passes through the third magnet 2014c, or otherwise when interacting with the third magnet 2014c, the third magnet 2014c may separate a portion of the second processed droplet 2062c including the magnetic beads 2056 from a portion of the second processed droplet 2062c including the protein of interest 2060p. The third side channel 2008c and/or the valve 2018c may then pull the portion of the second processed droplet 2062c including the magnetic beads 2056. The remaining portion (referred to as, a third processed droplet 2062d) may then travel toward the output valve 2016. The third processed droplet 2062d can be ejected as a product 2072 through the output valve 2016.

In some embodiments, one or more components described above may be integrated on a chip defined by external precision pumps and/or controlled valves. Pumps may include syringe pumps, gas driven pumps, peristaltic pumps, etc. Valves may include pneumatic valves, Quake vales, magnetic valves, plunger driven valves, etc. In some embodiments, input solution (e.g., the droplet 2060, reagents, etc.) containing a large number of different NA templates may be pumped into a junction with a pumped oil carrier. In some embodiments, the microfluidic system 2000 may encapsulate the input solution into droplets with limiting dilution to ensure that there is a single template in the droplet. In some embodiments, the microfluidic system 2000 may encapsulate surface functionalized magnetic beads (e.g., the magnetic beads 2056) and cell-free translation (and/or transcription) reagents (e.g., cell-free translation (and/or transcription) reagents 2058) in the droplets (e.g., the droplet 2060). After forming the droplet, the microfluidic system 2000 (e.g., the incubation region 2012) can incubate the mixture (e.g., the droplet 2060), allowing protein (e.g., the protein of interest 2060p) to form and subsequently bind to the functionalized magnetic bead. For example, the protein with a His-tag may be formed, and the magnetic beads may have a nickel moiety to bind to the tag. The magnetic beads retracted by the magnet, and a remaining portion may be pumped into waste by, for example, opening a first waste valve. The microfluidic system 2000 may pull the magnetic bead part of the droplet, wash the same by activating a washing reagent pump and opening a second waste valve, then elute the protein from the bead (e.g., with imidazole solution for the His-tag) by activating an elution pump and a third waste valve. Finally, the microfluidic system 2000 can dispense the protein by opening a product valve.

In some embodiments, one or more nozzles may be used to introduce the oil 2050 and/or the reagent, by for example, firing the nozzle. In some embodiments, one or more pumps (e.g., external pumps such as syringe, peristaltic etc., or internal pumps such as piezo, inertial, etc.) may be used to introduce the oil 2050 and/or the reagent, by for example, firing the nozzle.

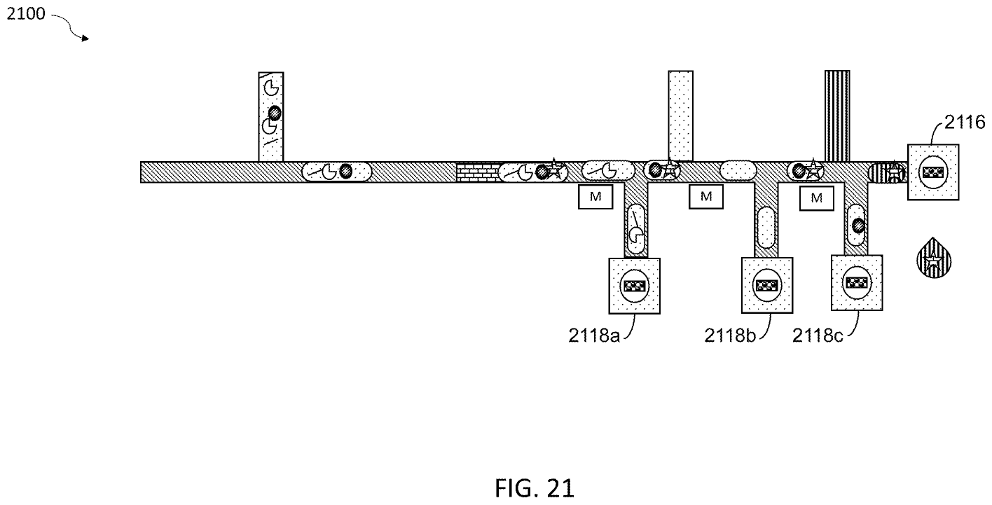

FIG. 21 shows top views of examples of microfluidic systems 2100 according to various embodiments. In some embodiments, as opposed to the valves described with respect to the microfluidic system 2000, the microfluidic system 2100 may alternatively include actuators. For example, the microfluidic system 2100 may include a first actuator 2118a, a second actuator 2118b, a third actuator 2118c (collectively referred to as actuators 2118), and an output actuator 2116. In some embodiments, for example when the microfluidic system 2100 is an integrated on-a-chip design, the microfluidic system 2100 may include various actuators, nozzles, and/or pumps. In some embodiments, the microfluidic system 2100 may include one or more actuators may be used to drive fluid flow through the channel and/or through a set of independent fluidic networks. For example, actuators including an inertial pump may be used to drive fluid flow through the channel. For example, an integrated inertial pump may be used to drive input fluids to control flows in the microfluidic chip. For example, the inertial pump may include, for example, a TIJ-bubble actuator. For example, actuator may include a TIJ driver. For example, a piezo-driven actuator may be used to drive fluid flow through the channel. For example, the inertial pump may be piezo-driven, or otherwise include any other displacement methods.

Completely-integrated design can work similarly to external driven pumps and may include a large number of different NA templates and encapsulate them into droplets with limiting dilution to ensure that there is a single template in this droplet. Concurrently, the microfluidic system 2100 may encapsulate surface functionalized magnetic beads and cell-free translation (and/or transcription) reagents in the same droplets. After forming the droplet, the microfluidic system 2100 can incubate the mixture, allowing protein to form and subsequently bind to the functionalized magnetic bead. For example, the microfluidic system 2100 may form the protein with a his tag, and the magnetic beads may have a nickel moiety to bind to the tag. The microfluidic system 2100 then can pull the magnetic bead part from the droplet, wash it, elute the protein from the bead (e.g. with imidazole solution for the His tag), and dispense the protein.

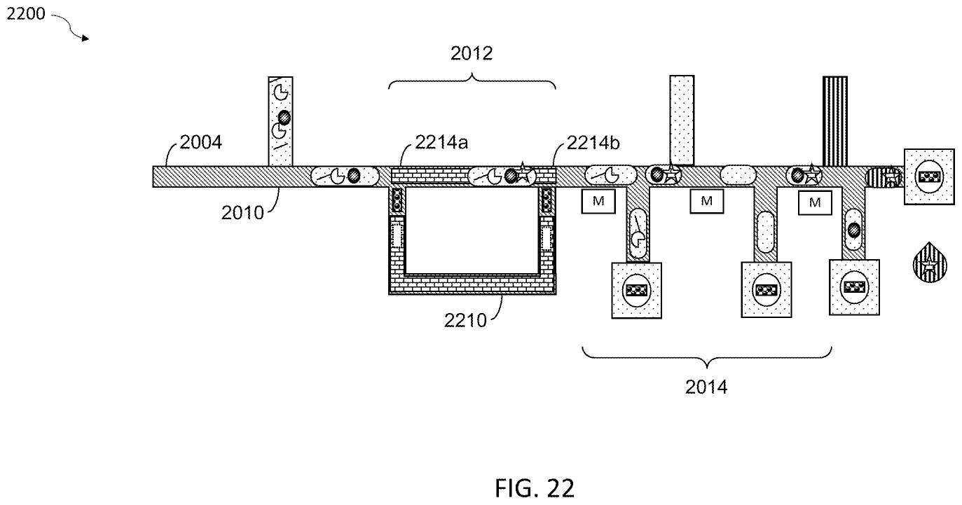

FIG. 22 shows top views of examples of microfluidic systems 2200 according to various embodiments. In some embodiments, the incubation region 2012 may include an incubation loop 2210. As shown in FIG. 22, the incubation loop 2210 may be disposed upstream of the magnetic actuation region 2014, and downstream of the junction. The incubation loop 2210 may include one or more droplets therein at the same time. In some embodiments, the incubation loop 2210 may include sensors. The sensors may be situated proximate the channel 2004. For example, the sensors may be or include an impedance sensor, a magnetic sensor, a giant magnetoresistance (GMR) sensor, an optical sensor, a sorting sensor, or any other sensor. Such a sensor may be to track a droplet location to sync a pump zone firing to avoid droplet destruction.

In some embodiments, the microfluidic system 2200 may include actuators situated proximate the channel 2004 and/or the incubation loop 2210. For example, the microfluidic system 2200 may include a first actuator situated proximate to a first junction 2214a between the incubation loop 2210 and the channel 2004, and a second actuator proximate to a second junction 2214b between the incubation loop 2210 and the channel 2004. In some embodiments, the first actuator and/or the second actuator may include a resistor. In some embodiments, the actuators may be or include an inertial pump. In some embodiments, the actuators may be or include a TIJ driver/nozzle. In some embodiments, the actuators may be or include a piezo-driven actuator.

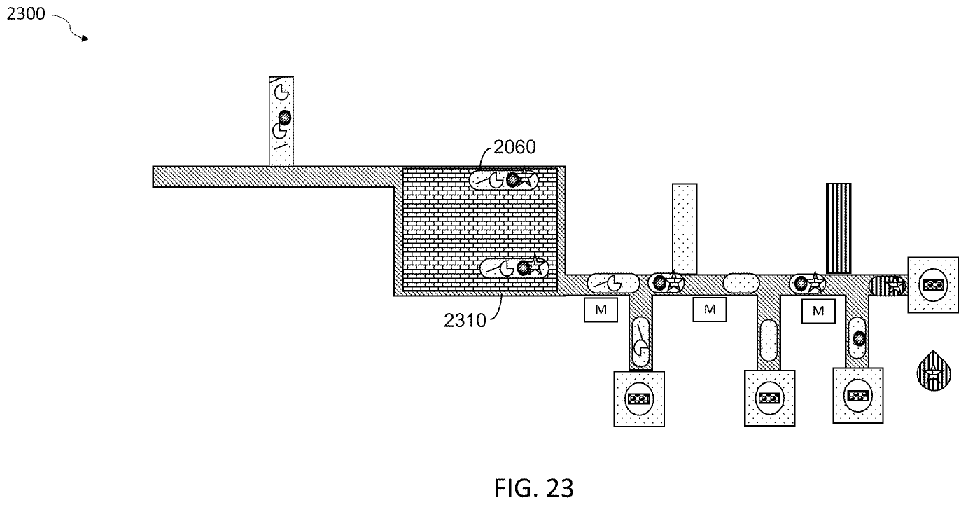

FIG. 23 shows top views of examples of microfluidic systems 2300 according to various embodiments. The microfluidic system 2300 may include an incubation chamber 2310. In some embodiments, the incubation chamber 2310 may be substantially identical to the incubation reservoir described with respect to FIG. 19 to FIG. 20. In some embodiments, the incubation chamber 2310 may include a plurality of droplets (e.g., the droplet 2060).

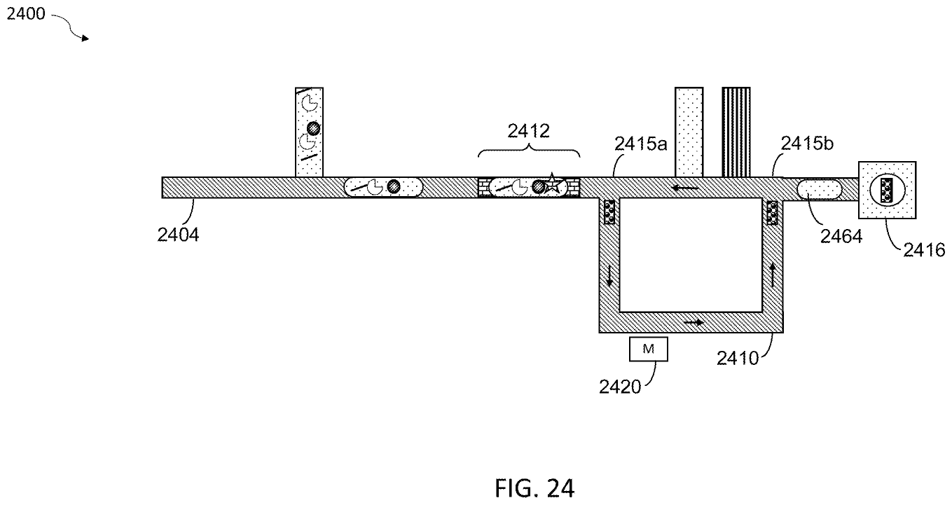

FIG. 24 shows top views of examples of microfluidic systems 2400 according to various embodiments. The microfluidic system 2400 may include a channel 2404, an incubation region 2412, a washing loop 2410, and an output actuator 2416. The washing loop 2410 may include an inlet of the loop 2415a, an outlet of the loop 2415b, and a magnet 2420. The washing loop 2410 may be disposed downstream of the incubation region 2012.

In some embodiments, the washing loop 2410 may repeat washing incubated droplets. For example, a first incubated droplet may be introduced into the washing loop 2410 at the inlet of the loop 2415a, flow through the washing loop 2410 while being washed with washing reagents, and be ejected into the channel 2404 at the outlet of the loop 2415b. In some embodiments, the first incubated droplet that has been washed may flow backward through the channel 2404 and can be re-introduced into the washing loop 2410.

In some embodiments, the microfluidic system 2400 may include actuators situated proximate the channel 2004 and/or the washing loop 2410. For example, the microfluidic system 2400 may include a first actuator situated proximate to the inlet of the loop 2415a between the washing loop 2410 and the channel 2404, and a second actuator proximate to the outlet of the loop 2415b between the washing loop 2410 and the channel 2404. In some embodiments, the first actuator and/or the second actuator may include a resistor. In some embodiments, the actuators may be or include an inertial pump. In some embodiments, the actuators may be or include a TIJ driver/nozzle. In some embodiments, the actuators may be or include a piezo-driven actuator.

The microfluidic system 2400 may eject a fluid/droplets through the output actuator 2416 into a multiwell plate. For example, the microfluidic system 2400 may eject the washing reagent 2464 into a junk well of a multiwell plate. For example, the microfluidic system 2400 may eject an incubated droplet that includes protein into a well of a multiwell plate for further processing. In some embodiments, a target proteins may be sequenced after being isolated.

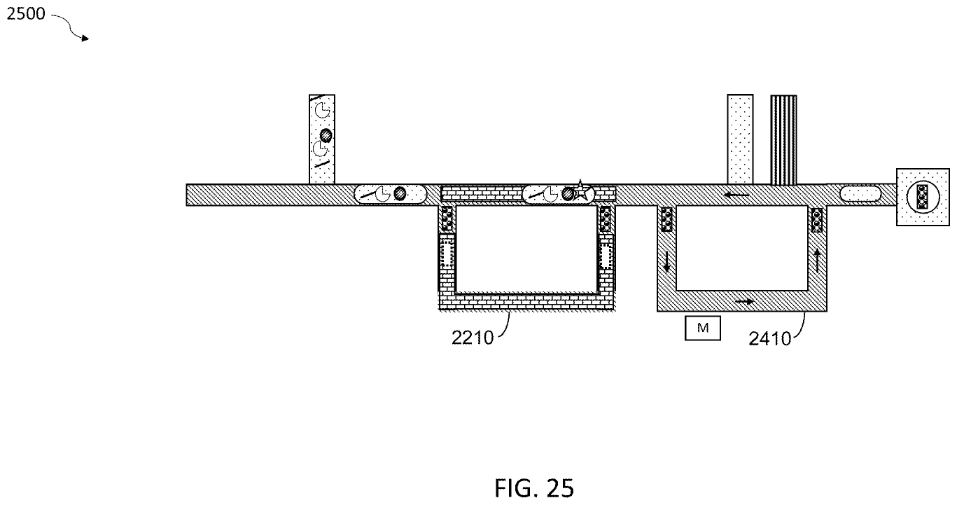

FIG. 25 shows top views of examples of microfluidic systems 2500 according to various embodiments. The microfluidic system 2500 may include a plurality of loop-based incubation, washing, and/or elution regions. For example, as shown in FIG. 25, the microfluidic system 2500 may include the incubation loop 2210 and the washing loop 2410.

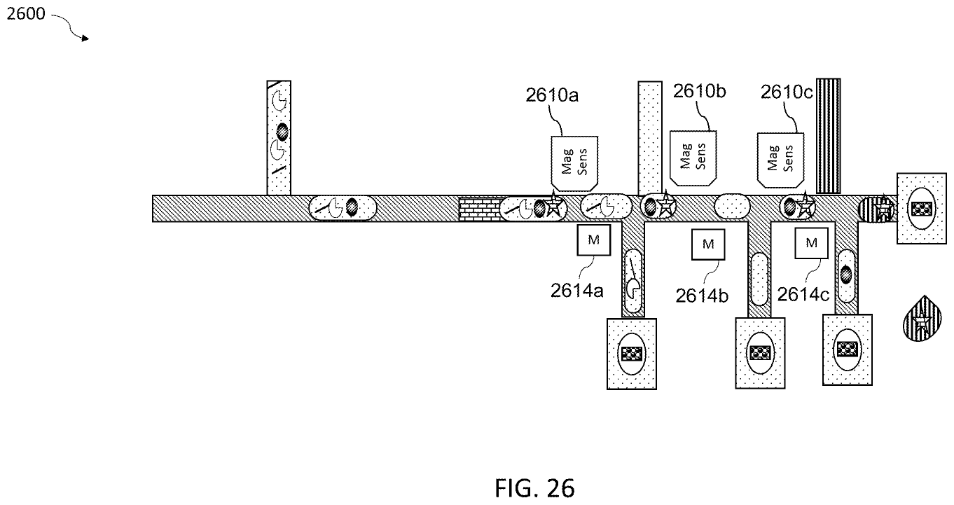

FIG. 26 shows top views of examples of microfluidic systems 2600 according to various embodiments. The microfluidic system 2600 may include a plurality of sensors for various purposes. In some embodiments, the microfluidic system 2600 may include magnetic sensors to identify drop positions. For example, the microfluidic system 2600 may include a first magnetic sensor 2610a, a second magnetic sensor 2610b, and a third magnetic sensor 2610c (collectively, magnetic sensors 2610). In some embodiments, the magnetic sensors 2610 may be a giant magnetoresistance sensor (GMR) sensor. In some embodiments, the sensors may be integrated into a silicon underlayer as a thin film GMR.

In some embodiments, the magnetic sensors 2610 may be disposed adjacent to a respective magnet. For example, the first magnetic sensor 2610a may be disposed adjacent to a first magnet 2614a, the second magnetic sensor 2610b may be disposed adjacent to a second magnet 2614b, and the third magnetic sensor 2610c may be disposed adjacent to a third magnet 2614c. The magnetic sensors 2610 and/or the magnets 2614 allows for observation of change of current in an electromagnet thereby detecting magnetic beads.

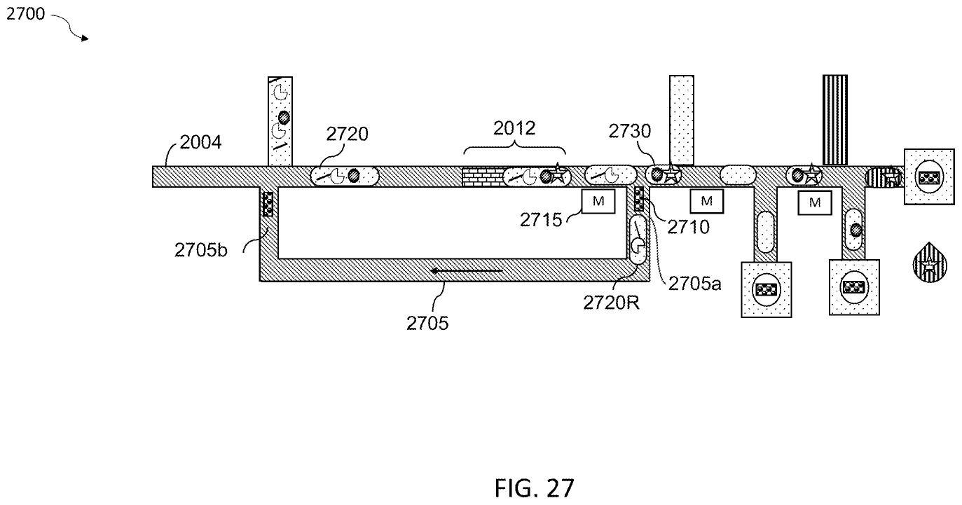

FIG. 27 shows top views of examples of microfluidic systems 2700 according to various embodiments. The microfluidic system 2700 may include one or more recycling loops. The recycling loop may include an inlet situated downstream of the incubation region and an outlet situated upstream of the input reservoir (or a channel/network connected thereto). For example, the microfluidic system 2700 may include a recycling loop 2705, which may include an inlet 2705a and an outlet 2705b.

The recycling loop 2705 may be to recycle or otherwise re-use at least a portion of a droplet 2720. For example, when incubation is complete on a droplet 2720, a magnet 2715 and a recycle actuator 2710 can sense, detect, and pull a portion of the droplet for recycle. For example, the magnet 2715 can sense or detect a recycled droplet 2720R or at least a portion thereof, and the recycle actuator 2710 can pull the recycled droplet 2720R into the recycling loop 2705, through the inlet 2705a. Meanwhile, a processed droplet 2730 can continue flowing through the channel 2004. In some embodiments, although not depicted, the recycling loop 2705 and/or the channel 2004 may include one or more reservoirs to form the droplet 2720 with the recycled droplet 2720R. For example, as shown in FIG. 27, when the recycled droplet 2720R does not include magnetic beads, the channel 2004 and/or the recycling loop 2705 may include a reservoir for magnetic beads, so as to form the droplet 2720 with the recycled droplet 2720R (e.g., adding a magnetic bead into the recycled droplet 2720R).

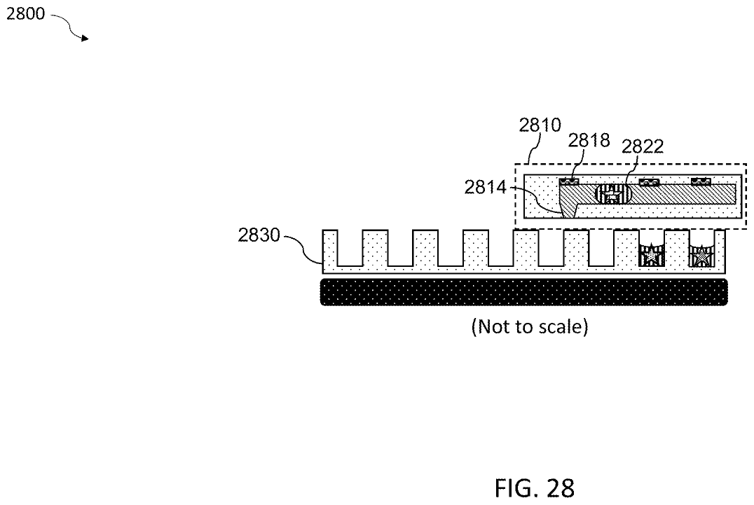

FIG. 28 shows cross-sectional views of examples of fluid dispensing systems 2800 according to various embodiments. The fluid dispensing system 2800 may include a dispenser 2810 and a multiwell plate 2830. The dispenser 2810 may include an output 2814, an actuator 2818, and a product 2822. The dispenser 2810 can eject the product 2822 into a well of the multiwell plate 2830 through the output 2814, by the actuator 2818. In some embodiments, the dispenser 2810 may eject one product 2822 into a well at a time. In some embodiments, the dispenser may eject a different product into a different well. In some embodiments, the dispenser may eject the product 2822 into a pre-determined well of the multiwell plate 2830. For example, a desired droplet may be ejected into a specific well or wells of the multiwell plate 2830 (e.g., microwell plate, nanowell plate, etc.). For example, a single droplet may be dedicated to a well. In some embodiments, the actuator 2818 of the dispenser 2810 may eject the product 2822 only when the actuator 2818 and/or the dispenser 2810 (or the fluid dispensing system 2800) has received a signal. The multiwell plate 2830 with the product 2822 may be delivered to a user for downstream processing. In some embodiments, the dispenser 2810 may eject the product 2822 into a junk well or wells.

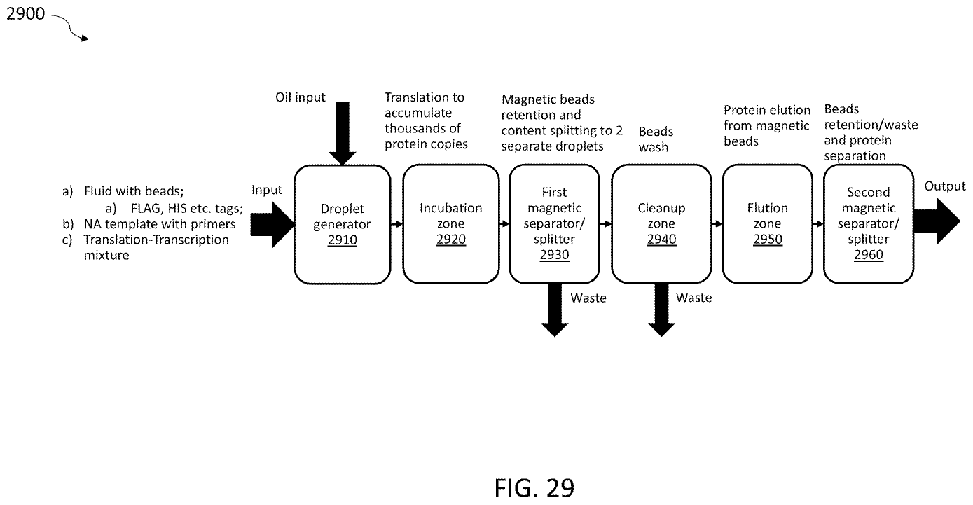

FIG. 29 shows a schematic diagram of examples of microfluidic systems 2900 according to various embodiments. The example microfluidic system 2900 may include a droplet generator 2910, an incubation zone 2920, a first magnetic separator/splitter 2930, a cleanup zone 2940, an elution zone 2950, and a second magnetic separator/splitter 2960. In some embodiments, fluid with beads (e.g., FLAG, HIS, etc. tags), NA template with primers, and/or translation-transcription mixture may be introduced as an input into the droplet generator 2910 through a first channel, while an oil (or immiscible fluid) may be introduced into the droplet generator 2910 through a second channel. The droplet generator 2910 then can generate a droplet with the input, encapsulated by the oil. The generated droplets can enter the incubation zone 2920, which can incubate the droplets and accumulate a plurality of (e.g., thousands of) protein copies. The incubated droplets including protein can be split to two separate droplets by the first magnetic separator/splitter 2930. In some embodiments, the first magnetic separator/splitter 2930 eject a portion of the incubated droplets that do not include the protein into waste, while introducing the other portion of the incubated droplets that includes the protein (referred to as the first processed droplet) into the cleanup zone. The cleanup zone 2940 can wash beads in the first processed droplet with washing reagents. The washed droplet can enter the elution zone 2950 where elution reagents elute the protein from the magnetic beads, while the washing reagents can be ejected to waste. The second magnetic separator/splitter 2960 can separate the magnetic beads from the protein, and the protein can be ejected into an output for downstream processing.

In some embodiments, the example microfluidic system 2000 may operate with various components and/or reagents. For example, the oil phase may be silicone oil (e.g., 1, 3, 5 or 10 cSt), mineral oil, or fluorinated hydrocarbons. The aqueous phase may be concentrated to a state of limiting dilution for the nucleic acid template, so in a volume of one droplet 1 copy of a template is found for ˜37% of the droplets. That is statistically 37% of the droplets containing 1 template, but all droplets may contain reagents for cell free protein synthesis and magnetic beads for purification of synthesized proteins. In some embodiments, when the droplets enter the incubation region, which can be kept at optimal temperature for the translation (and/or transcription) reaction of the template into the protein. In some embodiments, a set of primers and a polymerase, and/or dNTPs may be provided in the aqueous mixture. The incubation region may be thermos-cycled to amplify the single DNA template found in the aqueous droplets. This can increase the overall rate of production of the protein. In some embodiments, the translation (and/or transcription) reagent set may be or include wheat germ extract. In this case, in 10 pl drop, around 70,000 molecules/s can be produced. In some embodiments, the droplet may be kept in the incubation region for a sufficiently long time (e.g. 100 s of milliseconds) to produce sufficient amount of molecules for downstream analysis. In some embodiments, the template may include a region that encodes for a tag in the protein (e.g. His Tag, or FLAG tag) so that it can bind to an affinity probe on magnetic beads. As the proteins are produced, they can bind to the magnetic beads. When the incubation is complete, the droplets can enter the first magnet region. The motion of the droplet past the magnet can split the droplet into two parts. The first part that has the magnetic beads and the attached protein, and the second part is the remaining reagents. The second part may be shuttled to waste by a side channel with an ejector. The first part may be mixed with a wash buffer from a side channel, diluting the contents of the drop as to wash them. In some embodiments, magnetic bead separation may be repeated with a second region, where a magnet first breaks the droplet in half, with the portion without beads going to waste from a side outlet, and then the magnet releasing the droplet, allowing it to travel forward in the main channel. In some embodiments, several washing may be performed in series to achieve a sufficiently pure product. In some embodiments, the droplet with magnetic beads may be mixed with an elution buffer from a side channel. An example elution buffer may be 200 mM imidazole solution for HIS Tag capture. This can cause the coupling of the tag on the protein to be released from the affinity partner on the bead. In some embodiments, the droplet may be again moved over the magnet zone, where the droplet is split in half: one with the magnet beads and one without. In some embodiments, the part without the magnet beads (but with the protein of interest) may be pumped to the outlet and ejected out of the nozzle into a well of a multiwell plate. Meanwhile, the magnet may be turned off, releasing the part of the droplet with magnetic beads, which may be shuttled to waste.

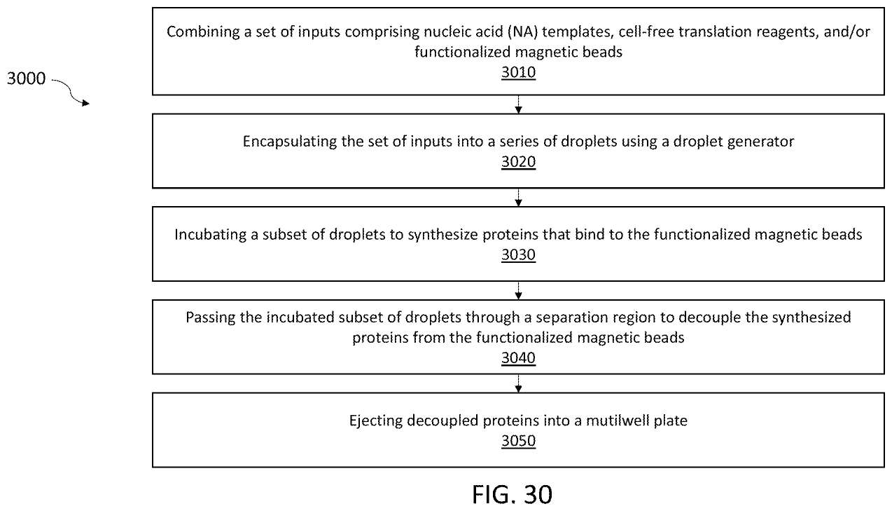

FIG. 30 shows an example flowchart of a method 3000 according to various embodiments. The method 3000 may be performed using any of the microfluidic systems described herein or any components thereof. The method 3000 may include combining a set of inputs from one or more input reservoirs of a microfluidic device, the one or more input reservoirs (i) feeding into a main channel of the microfluidic device, and (ii) including nucleic acid (NA) templates, cell-free translation (and/or transcription) reagents, and functionalized magnetic beads, each NA template coding for a different variant of the protein. The method 3000 may include encapsulating, using oil from an oil reservoir of the microfluidic device, the set of inputs into a series of droplets using a droplet generator of the microfluidic device such that each droplet in a subset of the series of droplets includes only one NA template. The method 3000 may include incubating, at an incubation zone of the main channel, the subset of droplets to synthesize proteins that bind to the functionalized magnetic beads. The method 3000 may include passing, via the main channel, the incubated subset of droplets through a separation region of the microfluidic device to decouple the synthesized proteins from the functionalized magnetic beads. The method 3000 may include ejecting decoupled proteins out of the microfluidic device and into wells of a mutilwell plate such that each well includes one of the variants of the protein.

In some embodiments, the method 3000 may include, when passing the incubated subset of droplets through the separation region, passing the incubated subset of droplets to a first magnet region of the microfluidic device to split each droplet into (i) a first part including magnetic beads with proteins attached thereto, and (ii) a second part including reagents.

In some embodiments, the method 3000 may include, when passing the incubated subset of droplets through the separation region, mixing the first part with a wash buffer from a first side channel of the microfluidic device, and/or passing the first part mixed with the wash buffer to a second magnet region to further purify the functionalized magnetic beads with proteins attached thereto from reagents.

In some embodiments, the method 3000 may include, when passing the incubated subset of droplets through the separation region, mixing the first part with an elution buffer from a second side channel of the microfluidic device to release proteins from the magnetic beads.

In some embodiments, the method 3000 may include using a magnetic zone to separate the magnetic beads from the proteins released from the magnetic beads, wherein the magnetic zone includes at least one of the first magnet region, the second magnet region, or a third magnet region, and ejecting the released proteins into the wells of the multiwell plate via a nozzle of the microfluidic device.

For example, as shown in FIG. 30, the method 3000 may begin by combining a set of inputs including nucleic acid (NA) templates, cell-free translation (and/or transcription) reagents, and/or functionalized magnetic beads (block 3010). In response to generation of the set of inputs, and/or the set of inputs being introduced to a droplet generator, the method 3000 may continue to encapsulating the set of inputs into a series of droplets using a droplet generator (block 3020). In response to generation of the droplet and/or the droplet being introduced into an incubation region, the method 3000 may continue to incubating a subset of droplets to synthesize proteins that bind to the functionalized magnetic beads (block 3030). In response to completion of the incubation, the method 3000 may continue to passing the incubated subset of droplets through a separation region to decouple the synthesized proteins from the functionalized magnetic beads (block 3040). In some embodiments, the method 3000 may include washing the droplets with washing reagents. In some embodiments, the method 3000 may include eluting the droplets with elution reagents. In some embodiments, the method 3000 may include using one or more magnets to separate the protein from the droplets. In some embodiments, the method 3000 may include ejecting at least a portion of the droplets to waste. In response to completion of the separation and/or the protein decoupling, the method 3000 may continue to ejecting decoupled proteins into a mutilwell plate (block 3050). In some embodiments, the method 3000 may include recycling at least a portion of the droplets.

In some embodiments, the method 3000 may be used to process cells including a nucleic acid encoding for production of a different product. For example, the droplet may be kept in the incubation region for a sufficiently long time (e.g. 100 s of milliseconds) to produce sufficient amount of molecules for downstream analysis. The template may include a region that encodes for a tag in protein (e.g. His Tag, or FLAG tag) so that it can bind to an affinity probe on magnetic beads. As the proteins are produced, they bind to the magnetic beads using the appropriate interaction molecules. The droplet then moves out of the incubation zone and passes a first magnet region. The motion of the droplet past the magnet splits the droplet into two parts. Each template may encode for a different variant of the protein.

The disclosure can be performed with various alternative components and/or various alternative methods. Peptide libraries are widely used in immunotherapy, vaccine development, drug discovery and proteomics research. Peptide-based drugs can be safer and more specific alternatives to small molecules. Identifying new peptide therapeutics can require increased synthesis scale and peptide structure elucidation after screening, which can be challenging when increasing the library size. Although advances in polynucleotide synthesis has made the production of large scale DNA libraries economical, the use of these DNA libraries to produce large ordered peptide libraries has not scaled to the same extent due, in part, to the limited scalability of peptide libraries from in vivo production systems. For example, generating a peptide library in vivo may involve cloning synthetic DNA constructs into plasmids, introducing the plasmids into living cells, in parallel, and then waiting for the cells to generate sufficient peptide for downstream analysis. Many peptides may induce toxic effects in living cells at higher concentrations.

Cell-free protein synthesis (CFPS) has the potential to overcome limits of the current in vivo production systems, particularly for production of toxic and complex proteins, and for high-throughput production. Because the half-life of CFPS reaction is relatively short, there is a desire to further reduce the cost and increase product yield of CFPS.

Research and clinical laboratories are under pressure to improve peptide synthesis while reducing costs and increasing throughput. The systems use for peptide engineering are transitioning to simpler microfluidic-based devices and systems, including devices and systems for library generation. Although advances in polynucleotide synthesis has made the production of large scale DNA libraries economical, the use of these DNA libraries to generate large-scale ordered peptide libraries has not been realized to the same extent due, in part, to in vivo production systems. For example, generating a peptide library in vivo may involve cloning synthetic DNA constructs into plasmids, introducing the plasmids into living cells, in parallel, and then waiting for the cells to generate sufficient peptide for downstream analysis. Many peptides may induce toxic effects, be degraded by, or be derivatized in living cells, and each target peptide variant must be isolated from cellular proteins. Cell-free protein synthesis (CFPS) has the potential to overcome limits of the current in vivo production systems, particularly for production of toxic and complex proteins, and for high-throughput production. Because the half-life of CFPS reaction is relatively short, there is a desire to increase product yield of CFPS and further reduce the cost, however.

Devices for synthesizing and dispensing a peptide in a droplet, in accordance with the present disclosure, may reduce the time and labor spent preparing peptide libraries by automating parallel synthesis and isolation of a single peptide variant from a template. These devices, which allow for efficient synthesis and isolation of a peptide produced by CFPS may increase peptide yield because fewer target peptides are lost during purification as compared with the purification process of in vivo systems. Devices in accordance with the present examples may have particular utility on-demand protein library generation by reducing the volume of sample and reagents compared to in vivo peptide synthesis, and/or by improving the speed, scale, and coverage of peptides which is advantageous for Identifying new peptide therapeutics and/or organism-wide gene product analysis. For example, a device as described below may dispense a droplet containing a peptide produced by CFPS using a single template may automate library generation, and such devices may efficiently dispense millions of fluid droplets organized for efficient screening and elucidation of positive hits via template sequencing, which has reduced barriers to access than peptide sequencing.

In one embodiment, a device may include a microfluidic network supported by a substrate comprising a main channel. The device may include a droplet ejector coupled to a distal end of the main channel to draw a first fluid through the main channel and dispense an eluted peptide droplet. The device may include a first inlet to suspend a droplet of a second fluid in the first fluid flowing in the main channel, the second fluid comprising cell-free peptide synthesis (CFPS) reagents and functionalized magnetic particles. The device may include an incubation zone to synthesize a peptide in the suspended droplet at a desired temperature. The device may include a first outlet fluidically downstream from the incubation zone and comprising a fluid actuator to draw a waste droplet comprising the CFPS reagents from the suspended droplet. The device may include a second inlet coupled to the main channel to merge an elution fluid droplet with the suspended droplet fluidically downstream from the first outlet. The device may include a magnet disposed in the substrate to extract the eluted-peptide droplet from the suspended droplet.

In some embodiments, the incubation zone may include a thermistor, a sensor, or a combination thereof. In some embodiments, the incubation zone may include a chamber or a loop-forming channel in fluidic communication with the main channel. In some embodiments, the first outlet may include a loop-forming channel and the magnet is disposed proximate to the loop-forming channel. In some embodiments, the substrate may include a sensor to identify a position of the suspended droplet in the main channel.

In one embodiment, a method include performing a series of cell-free peptide synthesis reactions in particle-laden droplets flowing in a microfluidic network. In some embodiments, the microfluidic network may include a proximal end receiving a carrier fluid, a distal end coupled to a droplet ejector, and an array of junctions between the proximal and distal ends. In some embodiments, the method include actuating the droplet ejector to flow the carrier fluid in the microfluidic network and suspend a reaction fluid into a plurality of droplets into the flowing carrier fluid at a first junction, the reaction fluid comprising cell-free peptide synthesis reagents (CFPS) including a set of nucleic acid templates each encoding a peptide, and a plurality of magnetic particles functionalized to capture a synthesized peptide, whereby a suspended droplet comprises a subset of both the nucleic acid templates and the magnetic particles. In some embodiments, the method may include flowing the suspended droplet through an incubation zone of the microfluidic network under conditions for CFPS. In some embodiments, the device may include actuating an inertial pump proximate to a second junction to separate a reagent fluid droplet from the suspended droplet. In some embodiments, the method may include merging an elution fluid droplet into the suspended droplet at a third junction fluidically downstream from the second junction to release synthesized peptides from the magnetic particles. In some embodiments, the method may include activating a magnetic field to immobilize the magnetic particles. In some embodiments, the method may include actuating the droplet ejector to dispense the synthesized peptides at a pre-determined location. In some embodiments, the method may include washing the functionalized magnetic particles of the suspended droplet fluidically downstream from the incubation zone. In some embodiments, the subset of nucleic acid templates may include a single nucleic acid template. In some embodiments, the method may include dispensing the reagent fluid droplet at a template-specific location and indexing the template-specific location with the pre-determined location. In some embodiments, the method may include identifying a position of the suspended droplet in the microfluidic network.

In one embodiment, a system may include a fluid supply assembly. The fluid supply assembly may include a first fluid reservoir to supply an encapsulating fluid, a second fluid reservoir to supply a second fluid immiscible with the encapsulating fluid, wherein the second fluid comprises cell-free peptide synthesis (CFPS) reagents and magnetic particles functionalized to capture synthesized peptides, and a third fluid reservoir to supply an elution fluid. In some embodiments, the system may include a dispenser including a microfluidic network supported by a substrate comprising a main channel in fluidic communication with the first fluid reservoir to flow the encapsulating fluid through a temperature controlled zone of the substrate downstream to a droplet ejector coupled to a distal end of the main channel, a plurality of inlets fluidically coupled to the fluid supply assembly and the main channel, wherein a first inlet suspends a droplet of the second fluid in the encapsulating fluid, and a second inlet disposed between the first inlet and the droplet ejector supplies the elution fluid to the suspended droplet, and a first outlet associated with a fluid actuator and a magnet to separate a waste droplet comprising the CFPS reagents from the suspended droplet, and a collection assembly to receive droplets dispensed by the droplet ejector. In some embodiments, the dispenser assembly may be incorporated into a mounting assembly. In some embodiments, the collection assembly may include an X-Y positioner to move a multi-well chamber relative to the mounting assembly. In some embodiments, the system may include a template collection assembly to receive the waste droplet from the first outlet. In some embodiments, the system may include an electric controller.

In one embodiment, a system may include a microfluidic network supported by a substrate comprising a main channel, a droplet ejector coupled to a distal end of the main channel, a first inlet to suspend a particle-laden droplet of a reaction fluid in a first fluid flowing in the main channel, the reaction fluid comprising cell-free peptide synthesis (CFPS) reagents and functionalized magnetic particles, an incubation zone to synthesize a peptide in the particle-laden droplet, a first outlet fluidically downstream from the incubation zone and comprising a fluid actuator, a second inlet coupled to the main channel and disposed fluidically downstream from the first outlet, a magnet disposed in the substrate proximate to the droplet ejector, and a controller operatively connected to the incubation zone, the droplet ejector, the fluid actuator, and the magnet, wherein the controller comprises a processing unit and a non-transient computer readable medium containing instructions that when executed, cause the processing unit to: control a temperature of the particle-laden droplet in the incubation zone, actuate the fluid actuator to remove a waste droplet comprising the CFPS reagents from the particle-laden droplet via the first outlet, apply a magnetic field to immobilize the particle-laden droplet, and actuate the droplet ejector to draw a first fluid through the main channel and suspend the particle-laden droplet via the first inlet, to draw an elution fluid into the main channel via the second inlet and merge the elution fluid and the particle-laden droplet, or to separate peptides from the immobilized particles and dispense the eluted peptides in a volume of fluid at a pre-determined location. In some embodiments, the incubation zone may include a thermistor operatively connected to the controller, and the instructions when executed, to cause the processing unit to activate the thermistor to heat the fluid in the incubation zone to a desired temperature. In some embodiments, the system may include a third inlet coupled to the main channel between the second inlet and the first outlet to merge a wash fluid droplet with the suspended droplet, a second outlet coupled to the main channel fluidically upstream of the second inlet, and a third outlet coupled to the main channel between the droplet ejector and the second inlet, and each of the first outlet, the second outlet, and the third outlet comprising an associated waste fluid ejector coupled to a distal end thereof. In some embodiments, the substrate may include a sensor operatively connected to the controller, and the instructions when executed, cause the processing unit to identify a position of the particle-laden droplet in the microfluidic network and to actuate the associated waste fluid ejector if the identified position is proximate the first outlet, the second outlet, or the third outlet, respectively. In some embodiments, each of the first outlet, the second outlet, and the third outlet may be associated with a first magnet, a second magnet and a third magnet, respectively, wherein each of the first, second, and third magnets may be operatively connected to the controller, and the instructions when executed, cause the processing unit to actuate the associated waste fluid ejector of the first, second, or third outlet when the associated magnet is activated.

An example method for dispensing an immunoreactive magnetic particle and immunoassay reporter in a fluid droplet is also described. The example method comprises actuating a droplet ejector coupled to a distal end of a microfluidic network to draw a fluid specimen comprising a suspension of immunoreactive magnetic particles and a reporter enzyme conjugate into a microfluidic channel, wherein if a target antigen is present, the target antigen, an immunoreactive magnetic particle, and the reporter enzyme conjugate form an enzyme-linked immunocomplex in the fluid specimen; applying a magnetic field in a region of the microfluidic channel upstream of the droplet ejector to immobilize the immunoreactive magnetic particles on a surface of the microfluidic channel; flowing a fluid comprising a signal precursor over the immobilized immunoreactive magnetic particles, whereby non-immobilized components of the fluid specimen are removed from the region; removing the magnetic field to release the immunoreactive magnetic particles; and dispensing a fluid droplet from the droplet ejector, the fluid droplet comprising a portion of the released immunoreactive magnetic particles and the signal precursor. The term “actuating” refers to or includes an act that causes an operation of the droplet ejector that causes the specimen fluid to flow into the microfluidic network. The phrase “applying a magnetic field” refers to or includes using a magnetic field to immobilize the immunoreactive magnetic particles on a surface of the microfluidic channel, and/or put the magnetic field into operation or position for the stated purpose/effect.

In some examples, the method comprises monitoring the dispensed fluid droplet for a signal catalyzed by the enzyme-linked immunocomplex, wherein detection of the signal indicates the target antigen is present in the fluid specimen. Monitoring may comprise detecting a chromogenic, fluorescent or chemiluminescent signal produced by the enzyme-linked immunocomplex.

In some examples, the fluid droplet is dispensed into a chamber filled with an oil immiscible with the fluid droplet. As used herein, an oil that is immiscible is incapable of being mixed with or attaining homogeneity with the fluid droplet. As used herein, “chamber” refers to or includes an enclosed and/or semi-enclosed region of a receptacle.

In some examples, a plurality of droplets are dispensed in the oil in a planar array or a three dimensional array. As used herein, “array” refers to or includes ordered series or arrangement, such as in row and/or columns.

In some examples, the method includes partitioning the dispensed droplets. For example, the plurality of droplets dispensed as an array may include droplets from more than one sample or populations of droplets specific for different analytes, and partitioning may allow samples to be grouped together within the array. As used herein, “partitioning” refers to or includes dividing the dispensed droplets into discrete populations. Partitioning may comprise dispensing a separation fluid droplet in the oil adjacent to a droplet of the plurality. As used herein, “separator fluid” refers to a fluid to delineate a boundary between dispensed droplets of the plurality.

An example system for dispensing an immunoreactive magnetic particle and immunoassay reporter in a fluid droplet is also described. The example system comprises: an immunoassay droplet generator comprising: a microfluidic network comprising: a microfluidic channel to receive a sample fluid comprising a set of enzyme-linked immunocomplexes comprising an immunoreactive magnetic particle, the microfluidic channel comprising a region to release capture the immunoreactive magnetic particles in response to a magnetic field; a signal precursor fluid channel in fluidic communication with the microfluidic channel at a position fluidically upstream of the region to releasably capture the immunoreactive magnetic particles and to introduce a signal precursor to the enzyme-linked immunocomplexes; and an outlet coupled to a distal end of the microfluidic channel comprising a droplet ejector to dispense an immunoassay droplet; and, an oil-filled chamber positioned to receive the dispensed immunoassay droplet, wherein the oil is immiscible with the immunoassay droplet. As used herein, “signal precursor” refers to and/or includes an immunoassay reporter as described above.

In some examples, the system comprises a signal detector. As used herein, “signal detector” refers to or includes a device or instrument designed to detect the presence of the signal generated by the immunoassay reporter, and/or emit a signal in response. The signal detector may comprise an optical sensor, contact image sensor (CIS), photodiode, LED, laser diode, photoresistor, photomultiplier tube (PMT), charge coupled device (CCD), complementary metal oxide semiconductor (CMOS), mirror or beamsplitter, lens, rod lens array, self-focusing lens array, optical filter, colorimeter, spectrophotometer, photodetector, fluorescence imager, plate reader, fluorescent microscope, confocal fluorimeter, camera, scanner, light or illumination source, such as an excitation source, laser, digital image processing system, or a combination thereof.

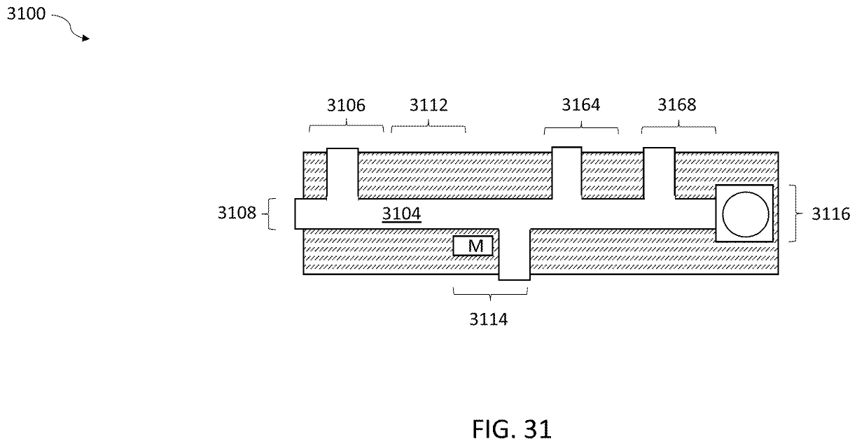

FIG. 31 shows top views of examples of devices 3100 for synthesizing and dispensing a peptide in a droplet according to various embodiments. The device 3100 may include an input region 3106 connected to an input reservoir or reagent reservoir providing NA templates, magnetic beads, and cell-free translation (and/or transcription) reagents. The device 3100 may include a fluid region 3108 connected to an immiscible fluid reservoir or oil reservoir providing an oil (or immiscible fluid). The device 3100 may include a channel 3104 (sometimes referred to as a fluidic network), a junction to generate a droplet, an incubation region 3112 where protein of interest is incubated, and a magnetic actuation region 3114 including a magnet disposed at the upstream side of a side channel. The device 3100 may include a separation region, including one or more side channels. The side channels may include a respective valve. The device 3100 may include a washing region 3164 providing a washing reagent and an elution region 3168 providing an elution reagent. The device 3100 may include an output region including an output valve to control ejection of a product. In some embodiments, the device 3100 may operate with external drivers.

The device 3100 may include a channel or a fluidic network. The device 3100 may include a set of reservoirs feeding the channel or the fluidic network, the set of reservoirs including an input reservoir disposed adjacent to the input region 3106 and an immiscible fluid reservoir disposed adjacent to the fluid region 3108. The device 3100 may include a droplet generator and/or a junction where the input region 3106 and the fluid region 3108 meets. The device 3100 may include the incubation region 3112 downstream of the junction. The device 3100 may include, downstream of the incubation region 3112, one or more magnetic actuation regions (e.g., the magnetic actuation region 3114), and one or more side channels.

The input region 3106 provides nucleic acid (NA) template. Each of the NA template may encode for a different variant of the protein. In the input region 3106, the NA template may combine with the cell-free translation (and/or transcription) reagent to produce protein in the incubation region 3112. The device 3100 may purify the protein using the magnetic bead and TIJ driven droplet manipulation at the washing region 3164. The device 3100 may elute the protein at the elution region 3168, and eject it into a multiwell plate at the output region 3116. The input region 3106 may provide the magnetic beads to the channel 3104. The magnetic beads may be with affinity to target protein. The input region 3106 may provide the cell-free translation (and/or transcription) reagents.

The incubation region 3112 may be substantially identical to the incubation reservoir and/or the incubation region descried with respect to FIG. 1 to FIG. 30. For example, the incubation region 3112 may be or include one or more incubation chambers or one or more incubation reservoirs/regions described with respect to FIG. 1 to FIG. 30.

The device 3100 may include the magnetic actuation region 3114. In some embodiments, the magnetic actuation region 3114 may be disposed downstream of the incubation region 3112. The magnetic actuation region 3114 may include a magnet and a side channel. In some embodiments, the magnet may be disposed at the upstream side of the side channel. In some embodiments, any of the magnet may be or include a permanent magnet. The side channel of the magnetic actuation region 3114 may form a separation region. The separation region may be disposed downstream of the magnetic actuation region 3114. In some embodiments, the device 3100 may include a plurality of magnetic actuation regions.

The device 3100 may include a washing region 3064 providing a washing reagent. The washing region 3064 may include a washing reagent reservoir to provide the washing reagent, for example downstream of at least one of the magnetic actuation region 3114 or the incubation region 3112. In some embodiments, the device 3100 may include a plurality of washing regions.

The device 3100 may include an elution region 3168 providing an elution reagent. The elution region 3168 may include an elution reagent reservoir to provide the elution reagent, for example downstream of at least one of the magnetic actuation region 3114 or the incubation region 3112.

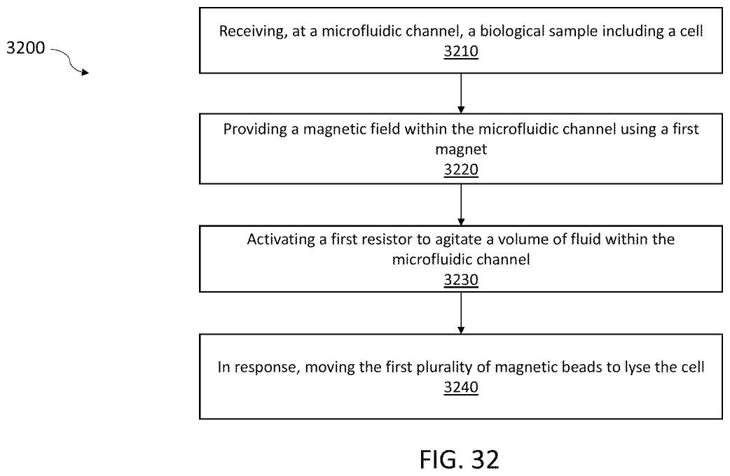

FIG. 32 shows an example flowchart of a method 3200 according to various embodiments. The method 3200 may include receiving, at a microfluidic channel, a biological sample including a cell (block 3210). In response to the receipt of the biological sample, the method 3200 may continue to providing a magnetic field within the microfluidic channel using a first magnet (block 3220). In some embodiments, based on an interaction between the magnetic field and at least a portion of the biological sample, the method 3200 may include splitting the biological sample into more than one portions. In response to providing the magnetic field, the method 3200 may continue to activating a first resistor to agitate a volume of fluid within the microfluidic channel (block 3230). In response to the agitation, the method 3200 may continue to moving the first plurality of magnetic beads to lyse the cell (block 3240).

Although specific examples have been illustrated and described herein, a variety of alternate and/or equivalent implementations may be substituted for the specific examples shown and described without departing from the scope of the present disclosure. This application is intended to cover any adaptations or variations of the specific examples discussed herein. Therefore, it is intended that this disclosure be limited only by the claims and the equivalents thereof.

The disclosure has been described above with reference to the various examples. However, it is to be understood that various modifications may be made in form and detail without departing from the scope of the disclosure as defined by the appended claims and their equivalents.

The various illustrative logical blocks, circuits, modules, routines, and algorithm steps described in connection with the embodiments disclosed herein can be implemented as electronic hardware, or combinations of electronic hardware and computer software. To clearly illustrate this interchangeability, various illustrative components, blocks, modules, and steps have been described above generally in terms of their functionality. Whether such functionality is implemented as hardware, or as software that runs on hardware, depends upon the particular application and design constraints imposed on the overall system. The described functionality can be implemented in varying ways for each particular application, but such implementation decisions should not be interpreted as causing a departure from the scope of the disclosure.

Moreover, the various illustrative logical blocks and modules described in connection with the embodiments disclosed herein can be implemented or performed by a machine, such as a general purpose processor device, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A control processor can synthesize a model for an FPGA. For example, the control processor can synthesize a model for logical programmable gates to implement a tensor array and/or a pixel array. The control channel can synthesize a model to connect the tensor array and/or pixel array on an FPGA, a reconfigurable chip and/or die, and/or the like. A general purpose processor device can be a microprocessor, but in the alternative, the processor device can be a controller, microcontroller, or state machine, combinations of the same, or the like. A processor device can include electrical circuitry configured to process computer-executable instructions. In another embodiment, a processor device includes an FPGA or other programmable device that performs logic operations without processing computer-executable instructions. A processor device can also be implemented as a combination of computing devices, e.g., a combination of a DSP and a microprocessor, a plurality of microprocessors, one or more microprocessors in conjunction with a DSP core, or any other such configuration. Although described herein primarily with respect to digital technology, a processor device may also include primarily analog components. For example, some or all of the algorithms described herein may be implemented in analog circuitry or mixed analog and digital circuitry. A computing environment can include any type of computer system, including, but not limited to, a computer system based on a microprocessor, a mainframe computer, a digital signal processor, a portable computing device, a device controller, or a computational engine within an appliance, to name a few.

The elements of a method, process, routine, or algorithm described in connection with the embodiments disclosed herein can be embodied directly in hardware, in a software module executed by a processor device, or in a combination of the two. A software module can reside in RAM memory, flash memory, ROM memory, EPROM memory, EEPROM memory, registers, hard disk, a removable disk, a CD-ROM, or any other form of a non-transitory computer-readable storage medium. An example storage medium can be coupled to the processor device such that the processor device can read information from, and write information to, the storage medium. In the alternative, the storage medium can be integral to the processor device. The processor device and the storage medium can reside in an ASIC. The ASIC can reside in a user terminal. In the alternative, the processor device and the storage medium can reside as discrete components in a user terminal.

While the above detailed description has shown, described, and pointed out novel features as applied to various embodiments, it can be understood that various omissions, substitutions, and changes in the form and details of the devices or algorithms illustrated can be made without departing from the spirit of the disclosure. As can be recognized, certain embodiments described herein can be embodied within a form that does not provide all of the features and benefits set forth herein, as some features can be used or practiced separately from others.

The herein described subject matter sometimes illustrates different components contained within, or connected with, different other components. It is to be understood that such depicted architectures are merely examples, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected,” or “operably coupled,” to each other to achieve the desired functionality, and any two components capable of being so associated can also be viewed as being “operably couplable,” to each other to achieve the desired functionality. Specific examples of operably couplable include but are not limited to physically mateable and/or physically interacting components and/or wirelessly interactable and/or wirelessly interacting components and/or logically interacting and/or logically interactable components.

The embodiments described herein have been described with reference to drawings. The drawings illustrate certain details of specific embodiments that implement the systems, methods and programs described herein. However, describing the embodiments with drawings should not be construed as imposing on the disclosure any limitations that may be present in the drawings.

It is important to note that the construction and arrangement of the devices, assemblies, and steps as shown in the various exemplary embodiments is illustrative only. Additionally, any element disclosed in one embodiment may be incorporated or utilized with any other embodiment disclosed herein. Although only one example of an element from one embodiment that can be incorporated or utilized in another embodiment has been described above, it should be appreciated that other elements of the various embodiments may be incorporated or utilized with any of the other embodiments disclosed herein.

### Selecting Cells

One aspect of the present disclosure provides method for selecting cells producing a specific product using a microfluidic device. The method comprising, consisting of, or consisting essentially of: packaging, into droplets, via a first fluidic network of the microfluidic device system described herein, cells that are in a cell suspension,; ejecting droplets into an incubation region of the microfluidic device; incubating the droplets in the incubation region at a temperature and for a period of time; pulling incubated droplets from the incubation region into a second fluidic network of the microfluidic device; screening for and in one embodiment detecting a desired product in a subset of the incubated droplets pulled from the incubation region; and sorting the incubated droplets based on whether the desired product is detected in the incubated droplets.

In some embodiments, the cells are packaged into the droplets. In some embodiments, each droplet includes no more than one cell from the cell suspension, and one or more suitable reagents from one or more reagent reservoirs of the microfluidic device described herein.

In some embodiments, the one or more reagents is a nucleic acid based barcode. A nucleic-acid based barcode is a short sequence of nucleotides (for example, DNA, RNA, or combinations thereof) that is used as an identifier for an associated molecule (e.g., a target molecule and/or target nucleic acid). A nucleic acid barcode can have a length of at least of about 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, or 100 nucleotides. A nucleic acid barcode can be in single- or double-stranded form. One or more nucleic acid barcodes can be attached, or “tagged,” to a target molecule and/or target nucleic acid. This attachment can be direct (for example, covalent or non-covalent binding of the barcode to the target molecule) or indirect (for example, via an additional molecule). The additional molecule can be a specific binding agent, such as an antibody (or other protein) or a barcode receiving adaptor (or other nucleic acid molecule). Target molecule and/or target nucleic acids can be labeled with multiple nucleic acid barcodes in combinatorial fashion, such as a nucleic acid barcode concatemer.

A nucleic acid barcode is used to identify a target molecule and/or target nucleic acids as being from a particular compartment; a discrete volume (e.g., the droplet described herein); having a particular physical property (e.g., affinity, length, sequence, etc.), or having been subject to certain treatment conditions. Target molecule and/or target nucleic acid can be associated with multiple nucleic acid barcodes to provide information about one or more desired features. Methods of generating nucleic acid-barcodes are disclosed, for example, in International Patent Application Publication No. WO/2014/047561.

In some embodiments, the nucleic acid barcode is optically detectable. Optically detectable barcodes are barcodes that can be detected with light or fluorescence microscopy. In some embodiments, the optical barcodes may comprise a sub-set of fluorophores or quantum dots of distinguishable colors from a set of defined colors. In some embodiments, beads are labeled with different ratios of dyes to form the set of defined colors from which the optical barcodes may be derived. For example, the beads may be polystyrene beads labeled with biotin conjugated dyes. Alternatively, the optical barcodes may be derived using a combination of optically detectable objects. For example, an optical barcode may be defined from a set of objects that can vary in size, shape, color, or any combination thereof that is distinguishable by light or fluorescence microscopy.

In some embodiments of the method disclosed herein, the one or more reagents can be a bead (e.g., a small hydrogel bead or other polymer structures such as e.g., poly-ethylene glycol di-acrylate beads or agarose beads). The one or more reagents can be a support. In some embodiments, each droplet-cell may be identified by assigning each individual cell with a unique barcode. This may be achieved by assigning a unique barcode to each individual cell or bead used in the method described herein and comprising a different microscale biological system. The individual bead may be pre-labeled with the unique barcode prior to packaging each individual bead with a cell in a droplet. Alternatively, the barcode may be introduced concurrently when packaging the cell in the droplet. For example, small hydrogel beads labeled with a barcode may be added to each individual droplet. In some embodiments, a second barcode may be similarly used to identify the one or more reporter elements in an individual droplet. The barcode may be a nucleic acid-based barcode. The barcode may be an optical barcode. The droplet can be an aqueous droplet in a water-in-oil emulsion.

In some embodiments, each cell is engineered to express a nucleic acid encoding a polypeptide of interest. In some embodiments of the method described herein, the cells in the cell suspension are packaged into a plurality of droplets. In some embodiments, each droplet of the plurality of droplet comprises a cell expressing a nucleic acid encoding a molecule that is different from the molecule expressed by a cell packaged by another droplet in the plurality of droplet.

In some embodiments of the method described herein, the cells may be eukaryotic cells, prokaryotic cells, or plant cells. In certain example embodiments, the cells may be naturally occurring. In certain example embodiments, the cells may be isolated from clinical isolates. Any suitably clinical isolate may be used in the present invention including, but are not limited to, a biopsy or other tissue sample, a blood sample, a saliva sample, and a urine sample. In certain example environments the cells may be isolated from an environmental sample. Environmental samples include, but are not limited to household/commercial/industrial surfaces (metal, wood, plastic), soil samples, and water samples (fresh and saline). In some embodiments, the cell is a eukaryotic cell or a prokaryotic cell. For example, the cell can be E. coli, wheat germ, Vibrio natriegens, Leishmania tarentolae, tobacco, HeLa, Pseudomonas putida, Streptomyces, Bacillus megaterium, Chinese hamster ovary (CHO), insect, Bacillus subtilis, yeast, archaeal, and rabbit reticulocyte.

In some embodiments, the cells may be engineered to comprise one or more mutation or exogenous gene. For example, the cell suspension can comprise a population of cells, where each cell can express a different genetic mutation or transgenes or a combination of genetic mutations or transgenes to be screened. Mutations may include gene knock-outs, gene knock-ins, transpositions, inversions, and/or one or more nucleotide insertions, deletions, or substitutions. The population of cells can then be screened using the method disclosed herein to assess an impact the one or more genetic mutations has on one or more product.

In some embodiments, the cells are microbial cells. In some embodiments, the microbial cells are bacterial cells. In certain other example embodiments, the microbial cells are fungal cells. Any type of microbial cell may be screened using the present assay.

In some embodiments, the method disclosed herein can be used to screen bacterial cells for biological functions. In some embodiments, the biological function is the production of one or more bacterial cell products (e.g., a product having antibiotic activity). In some embodiments, the bacteria may comprise different species of bacteria, different strains of the same species of bacteria, or a combination thereof. The bacteria may be isolated from a natural environment or a recombinant. For example, many existing antibiotics are produced by bacterial species commonly found in the soil. In some embodiments, the method disclosed herein can be used to screen natural or engineered bacteria for novel antibiotic activity.

This disclosure includes an example for how a design can be applied to a specific problem of Streptomyces strain engineering, namely developing (and/or selecting) Streptomyces strains that produce the desired product. Similar designs can be applied to antibody discovery (e.g., selecting hybridoma cells that produce desired antibodies via homogeneous proximity assays), and other drug discovery (e.g., observing how drugs influence sensor cells, cells that model disease pathways, and/or output a reporter when the pathways are altered).

As used herein, the term “Streptomyces” refers to a genus of gram-positive bacteria, from which a large number of antibiotics and other bioactive substances have been derived. Strains of Streptomyces are used industrially for production of small molecule drugs, such as rapamycin, avermectin, and tylosin, as well as proteins (e.g., phospholipase D). Some strains have a high innate protein secretion capacity, and thus are an ideal chassis for enzyme production. One aspect of the present disclosure provides, a method for screening an acellular sample for a specific product using a microfluidic device, the method comprising, consisting of, or consisting essentially of: packaging, into droplets, via a first fluidic network of the microfluidic device system described herein, the acellular sample; ejecting droplets into an incubation region of the microfluidic device; incubating the droplets in the incubation region at a temperature and for a period of time; pulling incubated droplets from the incubation region into a second fluidic network of the microfluidic device; screening for and detecting a desired product in a subset of the incubated droplets pulled from the incubation region; and sorting the incubated droplets based on whether the desired product is detected in the incubated droplets.

An acellular sample may include, but is not limited to, a cellular extract, a cellular fraction or sub-fraction (including nuclear or cytoplasmic fractions or isolated organelles), a cell-free system, and/or an in vitro solution, including but not limited to, an aliquot from an assay screen, probe or experimental solution or reaction. Cell-free systems may comprise a nucleic acid construct or set of nucleic acid constructs encoding a set of gene expression products. Each nucleic acid construct may comprise a different combination of regulatory elements and genes and/or ordering of regulatory elements and genes. Hence, the present disclosure may be used to screen a set of synthetic biology constructs for one or more target biological functions or to select an optimal construct architecture.

In some embodiments, the method disclosed herein comprise a cell-free protein synthesis (CFPS) system as described herein. Additional exemplary CFPS system can include cell extracts associated with specific organisms. Exemplary organisms and organelles for use as a source for CFPS include, for example, E. coli, Streptomyces, Saccharopolyspora spinosa, corn, chloroplasts, Myxococcus xanthus, lactic acid bacteria, and coryneform bacteria. In some embodiments, the CFPS can be packaged in the droplets described herein. In some embodiments, plasmids containing genes of interest are packaged in a droplet with or without a bead and including a plurality of attached barcode and a CFPS reagent. In that embodiment, each droplet receives approximately one barcoded sample and/or bead and no more than one plasmid. In some embodiments, the genes/plasmid are transcribed into mRNA, which is optionally reverse transcribed into cDNA. The mRNA or cDNA can then be tagged with the barcodes in a origin-specific manner (e.g., a distinct barcode for each emulsion droplet). A plasmid may contain more than one desired gene, such that multiple distinct mRNAs/cDNAs are generated in a droplet from a single plasmid. In that embodiment, each of the multiple distinct mRNAs/cDNAs can be labeled with the same barcode, thereby indicating the droplet from which they originated (e.g., origin-specific barcoding). In some embodiments, the mRNA is translated into polypeptides, which can also be tagged with the origin-specific barcodes, thereby origin-specifically barcoding the polypeptides and the mRNAs/cDNAs encoding the polypeptides with the same barcodes. Barcoded mRNAs/cDNAs and/or polypeptides can be pooled (e.g., by breaking the emulsion), and then sequenced to determine, the origin-specific transcript levels and quantification of parts.

The foregoing description of embodiments has been presented for purposes of illustration and description. It is not intended to be exhaustive or to limit the disclosure to the precise form disclosed, and modifications and variations are possible in light of the above teachings or may be acquired from this disclosure. The embodiments were chosen and described in order to explain the principals of the disclosure and its practical application to enable one skilled in the art to utilize the various embodiments and with various modifications as are suited to the particular use contemplated. Other substitutions, modifications, changes and omissions may be made in the design, operating conditions and arrangement of the embodiments without departing from the scope of the present disclosure as expressed in the appended claims.

### EXAMPLES

Sample implementations are disclosed below, in order to represent illustrative examples, which may be further modified, combined, constrained, etc. according to the entirety of this disclosure.

Example AA: a microfluidic system comprising: a set of reservoirs, the set of reservoirs comprising a reagent reservoir and an immiscible fluid reservoir; an incubation region having an inlet and an outlet; a set of independent fluidic networks, the set of independent fluidic networks comprising a first fluidic network and a second fluidic network that is not fluidically connected to the first fluidic network, wherein the first fluidic network (1) is fed by the set of reservoirs, (2) comprises a droplet generator, and (3) connects to the inlet of the incubation region, and wherein the second fluidic network is connected to the outlet of the incubation region.

Example AB: Example AA, wherein the droplet generator encapsulates reagents from the reagent reservoir into droplets.

Example AC: Example AA or Example AB, wherein the first fluidic network connects to the inlet of the incubation region via a fluid actuator.

Example AD: Example AC, wherein the fluid actuator is a thermal ink jet actuator.

Example AE: Any of Examples AA-AD, further comprising a sensing region for particle detection, the sensing region comprising impedance sensing electrodes.

Example AF: Any of Examples AA-AE, comprising multiple channels of droplet generation feeding into the incubation region.

Example AG: Any of Examples AA-AF, further comprising optical components for characterizing content of droplets.

Example AH: Any of Examples AA-AG, wherein desired droplets are ejected from the second fluidic network into wells of a microwell plate for downstream processing.

Example AL: Any of Examples AA-AH, wherein the set of reservoirs comprises magnetic beads.

Example AJ: Any of Examples AA-AI, wherein the incubation region comprises an incubation chamber.

Example AK: Any of Examples AA-AJ, wherein the incubation region comprises an incubation loop.

Example AL: Example AK, further comprising a first actuator situated proximate to a first junction between the incubation loop and the channel, and a second actuator proximate to a second junction between the incubation loop and the channel.

Example AM: Example AL, wherein the first actuator comprises a first resistor, and wherein the second actuator comprises a second resistor.

Example AN: Any of Examples AA-AM, further comprising one or more magnetic actuation regions downstream of the incubation region.

Example AO: Example AN, further comprising a separation region downstream of the one or more magnetic actuation regions, the separation region comprising one or more side channels.

Example AP: Any of Examples AA-AO, further comprising, downstream of the incubation region, a washing reagent reservoir and an elution reagent reservoir.

Example AQ: Any of Examples AA-AP, further comprising a recycling loop having an inlet situated downstream of the incubation region, and an outlet situated upstream of the set of reservoirs.

Example AR: Any of Examples AA-AQ, further comprising one or more actuators to drive fluids flow through the set of independent fluidic networks.

Example AS: Any of Examples AA-AR, further comprising using a measurement system to detect the cells prior to packaging the cells into the droplets, the measurement system comprising a pair of impedance electrodes.

Example AT: Any of Examples AA-AS, wherein packaging the cells into the droplets comprises using oil from an oil reservoir of the microfluidic device.

Example AU: Any of Examples AA-AT, wherein the one or more reagents assay the desired product.

Example AV: Any of Examples AA-AU, wherein sorting the incubated droplet comprises using optics to generate an excitation illumination and detect collection wavelengths.

Example BA: a method for selecting cells producing a specific product using a microfluidic device, the method comprising: packaging, into droplets, via a first fluidic network of the microfluidic device, cells that are in a cell suspension, wherein the cells are packaged into the droplets such that each droplet includes (1) no more than one cell from the cell suspension, and (2) one or more reagents from one or more reagent reservoirs of the microfluidic device; ejecting droplets into an incubation region of the microfluidic device; incubating the droplets in the incubation region at a temperature and for a period of time; pulling incubated droplets from the incubation region into a second fluidic network of the microfluidic device; detecting a desired product in a subset of the incubated droplets pulled from the incubation region; and sorting the incubated droplets based on whether the desired product is detected in the incubated droplets.

Example BB: Example BA, wherein each cell includes a nucleic acid encoding for production of a different product.

Example CA: a microfluidic system comprising: a channel; a set of reservoirs feeding the channel, the set of reservoirs comprising: an input reservoir comprising magnetic beads; and an oil reservoir; a droplet generator downstream of a junction between the channel and the input reservoir; an incubation region downstream of the junction; and downstream of the incubation region, one or more magnetic actuation regions, and one or more side channels.

Example CB: Example CA, wherein the incubation region comprises an incubation chamber.

Example CC: Example CA or Example CB, wherein the incubation region comprises an incubation loop.

Example CD: Example CC, further comprising a first actuator situated proximate to a first junction between the incubation loop and the channel, and a second actuator proximate to a second junction between the incubation loop and the channel.

Example CE: Example CD, wherein the first actuator comprises a first resistor, and wherein the second actuator comprises a second resistor.

Example CF: Any of Examples CA-CE, further comprising a separation region downstream of the one or more magnetic actuation regions, wherein the one or more side channels are part of the separation region.

Example CG: Any of Examples CA-CF, further comprising, downstream of the magnetic actuation regions, a washing reagent reservoir and an elution reagent reservoir.

Example CH: Any of Examples CA-CG, further comprising a recycling loop having an inlet situated downstream of the incubation region, and an outlet situated upstream of the input reservoir.

Example CI: Any of Examples CA-CH, further comprising one or more actuators to drive fluids flow through the channel.

Example CJ: Example CI, the one or more actuators comprising an inertial pump.

Example CK: Example CI, the one or more actuators comprising a thermal inkjet driver.

Example CL: Any of Examples CA-CK, the one or more actuators comprising a piezo-driven actuator.

Example CM: Any of Examples CA-CL, further comprising a permanent magnet at the magnetic region.

Example CO: Any of Examples CA-CM, further comprising one or more magnetic sensors situated proximate the channel.

Example CP: Example CO, wherein the one or more magnetic sensors comprises a giant magnetoresistance (GMR) sensor.

Example DA: a method comprising: combining a set of inputs from one or more input reservoirs of a microfluidic device, the one or more input reservoirs (i) feeding into a main channel of the microfluidic device, and (ii) comprising nucleic acid (NA) templates, cell-free translation (and/or transcription) reagents, and functionalized magnetic beads, each NA template coding for a different variant of the protein; encapsulating, using oil from an oil reservoir of the microfluidic device, the set of inputs into a series of droplets using a droplet generator of the microfluidic device such that each droplet in a subset of the series of droplets comprises only one NA template; incubating, at an incubation zone of the main channel, the subset of droplets to synthesize proteins that bind to the functionalized magnetic beads; passing, via the main channel, the incubated subset of droplets through a separation region of the microfluidic device to decouple the synthesized proteins from the functionalized magnetic beads; and ejecting decoupled proteins out of the microfluidic device and into wells of a mutilwell plate such that each well comprises one of the variants of the protein.

Example DB: Example DA, wherein passing the incubated subset of droplets through the separation region comprises passing the incubated subset of droplets to a first magnet region of the microfluidic device to split each droplet into (i) a first part comprising magnetic beads with proteins attached thereto, and (ii) a second part comprising reagents.

Example DC: Example DB, wherein passing the incubated subset of droplets through the separation region further comprises: mixing the first part with a wash buffer from a first side channel of the microfluidic device; and passing the first part mixed with the wash buffer to a second magnet region to further purify the functionalized magnetic beads with proteins attached thereto from reagents.

Example DD: Example DB, wherein passing the incubated subset of droplets through the separation region further comprises mixing the first part with an elution buffer from a second side channel of the microfluidic device to release proteins from the magnetic beads.

Example DE: Example DD, further comprising: using a magnetic zone to separate the magnetic beads from the proteins released from the magnetic beads, wherein the magnetic zone comprises at least one of the first magnet region, the second magnet region, or a third magnet region; and ejecting the released proteins into the wells of the multiwell plate via a nozzle of the microfluidic device.

Example EA: a device comprising: a microfluidic network supported by a substrate comprising a main channel; a droplet ejector coupled to a distal end of the main channel to draw a first fluid through the main channel and dispense an eluted peptide droplet; a first inlet to suspend a droplet of a second fluid in the first fluid flowing in the main channel, the second fluid comprising cell-free peptide synthesis (CFPS) reagents and functionalized magnetic particles; an incubation zone to synthesize a peptide in the suspended droplet at a desired temperature; a first outlet fluidically downstream from the incubation zone and comprising a fluid actuator to draw a waste droplet comprising the CFPS reagents from the suspended droplet; a second inlet coupled to the main channel to merge an elution fluid droplet with the suspended droplet fluidically downstream from the first outlet; and a magnet disposed in the substrate to extract the eluted-peptide droplet from the suspended droplet.

Example EB: Example EA, wherein the incubation zone includes a thermistor, a sensor, or a combination thereof.

Example EC: Example EA or Example EB, wherein the incubation zone comprises a chamber or a loop-forming channel in fluidic communication with the main channel.

Example ED: Any of Examples EA-EC, wherein the first outlet comprises a loop-forming channel and the magnet is disposed proximate to the loop-forming channel.

Example EE: Any of Examples EA-ED, the substrate comprising a sensor to identify a position of the suspended droplet in the main channel.

Example FA: a method comprising: performing a series of cell-free peptide synthesis reactions in particle-laden droplets flowing in a microfluidic network, wherein the microfluidic network comprises: a proximal end receiving a carrier fluid, a distal end coupled to a droplet ejector, and an array of junctions between the proximal and distal ends; actuating the droplet ejector to flow the carrier fluid in the microfluidic network and suspend a reaction fluid into a plurality of droplets into the flowing carrier fluid at a first junction, the reaction fluid comprising cell-free peptide synthesis reagents (CFPS) including a set of nucleic acid templates each encoding a peptide, and a plurality of magnetic particles functionalized to capture a synthesized peptide, whereby a suspended droplet comprises a subset of both the nucleic acid templates and the magnetic particles; flowing the suspended droplet through an incubation zone of the microfluidic network under conditions for CFPS; actuating an inertial pump proximate to a second junction to separate a reagent fluid droplet from the suspended droplet; merging an elution fluid droplet into the suspended droplet at a third junction fluidically downstream from the second junction to release synthesized peptides from the magnetic particles; activating a magnetic field to immobilize the magnetic particles; and actuating the droplet ejector to dispense the synthesized peptides at a pre-determined location.

Example FB: Example FA, comprising washing the functionalized magnetic particles of the suspended droplet fluidically downstream from the incubation zone.

Example FC: Example FA or Example FB, wherein the subset of nucleic acid templates consists of a single nucleic acid template.

Example FD: Any of Examples FA-FC, comprising dispensing the reagent fluid droplet at a template-specific location and indexing the template-specific location with the pre-determined location.

Example FE: Any of Examples FA-FD, comprising identifying a position of the suspended droplet in the microfluidic network.

Example GA: a system comprising: a fluid supply assembly comprising: a first fluid reservoir to supply an encapsulating fluid, a second fluid reservoir to supply a second fluid immiscible with the encapsulating fluid, wherein the second fluid comprises cell-free peptide synthesis (CFPS) reagents and magnetic particles functionalized to capture synthesized peptides; and a third fluid reservoir to supply an elution fluid; a dispenser assembly comprising: a microfluidic network supported by a substrate comprising a main channel in fluidic communication with the first fluid reservoir to flow the encapsulating fluid through a temperature controlled zone of the substrate downstream to a droplet ejector coupled to a distal end of the main channel; a plurality of inlets fluidically coupled to the fluid supply assembly and the main channel, wherein a first inlet suspends a droplet of the second fluid in the encapsulating fluid, and a second inlet disposed between the first inlet and the droplet ejector supplies the elution fluid to the suspended droplet; and a first outlet associated with a fluid actuator and a magnet to separate a waste droplet comprising the CFPS reagents from the suspended droplet; and a collection assembly to receive droplets dispensed by the droplet ejector.

Example GB: Example GA, wherein the dispenser assembly is incorporated into a mounting assembly.

Example GC: Example GB, wherein the collection assembly includes an X-Y positioner to move a multi-well chamber relative to the mounting assembly.

Example GD: Any of Examples GA-GC, comprising a template collection assembly to receive the waste droplet from the first outlet.

Example GE: Any of Examples GA-GD, comprising an electric controller.

Example HA: a system comprising: a microfluidic network supported by a substrate comprising a main channel; a droplet ejector coupled to a distal end of the main channel; a first inlet to suspend a particle-laden droplet of a reaction fluid in a first fluid flowing in the main channel, the reaction fluid comprising cell-free peptide synthesis (CFPS) reagents and functionalized magnetic particles; an incubation zone to synthesize a peptide in the particle-laden droplet; a first outlet fluidically downstream from the incubation zone and comprising a fluid actuator; a second inlet coupled to the main channel and disposed fluidically downstream from the first outlet; a magnet disposed in the substrate proximate to the droplet ejector, and a controller operatively connected to the incubation zone, the droplet ejector, the fluid actuator, and the magnet, wherein the controller comprises a processing unit and a non-transient computer readable medium containing instructions that when executed, cause the processing unit to: control a temperature of the particle-laden droplet in the incubation zone; actuate the fluid actuator to remove a waste droplet comprising the CFPS reagents from the particle-laden droplet via the first outlet; apply a magnetic field to immobilize the particle-laden droplet; and actuate the droplet ejector to draw a first fluid through the main channel and suspend the particle-laden droplet via the first inlet; to draw an elution fluid into the main channel via the second inlet and merge the elution fluid and the particle-laden droplet; or to separate peptides from the immobilized particles and dispense the eluted peptides in a volume of fluid at a predetermined location.

Example HB: Example HA, wherein the incubation zone comprises a thermistor operatively connected to the controller, and the instructions when executed, cause the processing unit to activate the thermistor to heat the fluid in the incubation zone to a desired temperature.

Example HC: Example HA or Example HB, comprising a third inlet coupled to the main channel between the second inlet and the first outlet to merge a wash fluid droplet with the suspended droplet, a second outlet coupled to the main channel fluidically upstream of the second inlet, and a third outlet coupled to the main channel between the droplet ejector and the second inlet, and each of the first outlet, the second outlet, and the third outlet comprising an associated waste fluid ejector coupled to a distal end thereof.

Example HD: Example HC, wherein the substrate comprises a sensor operatively connected to the controller, and the instructions when executed, cause the processing unit to identify a position of the particle-laden droplet in the microfluidic network and to actuate the associated waste fluid ejector if the identified position is proximate the first outlet, the second outlet, or the third outlet, respectively.

Example HE: Example HC, wherein each of the first outlet, the second outlet, and the third outlet are associated with a first magnet, a second magnet and a third magnet, respectively, wherein each of the first, second, and third magnets are operatively connected to the controller, and the instructions when executed, cause the processing unit to actuate the associated waste fluid ejector of the first, second, or third outlet when the associated magnet is activated.

What is claimed:

1. A microfluidic system comprising:
a set of reservoirs, the set of reservoirs comprising a reagent reservoir and an immiscible fluid reservoir;
an incubation region having an inlet and an outlet;
a set of independent fluidic networks, the set of independent fluidic networks comprising a first fluidic network and a second fluidic network that is not fluidically connected to the first fluidic network,
wherein the first fluidic network (1) is fed by the set of reservoirs, (2) comprises a droplet generator, and (3) connects to the inlet of the incubation region, and
wherein the second fluidic network is connected to the outlet of the incubation region.

2. The microfluidic system of claim 1, wherein the droplet generator encapsulates reagents from the reagent reservoir into droplets.

3. The microfluidic system of claim 1, wherein the first fluidic network connects to the inlet of the incubation region via a fluid actuator.

4. The microfluidic system of claim 3, wherein the fluid actuator is a thermal ink jet actuator.

5. The microfluidic system of claim 1, further comprising a sensing region for particle detection, the sensing region comprising impedance sensing electrodes.

6. The microfluidic system of claim 1, comprising multiple channels of droplet generation feeding into the incubation region.

7. The microfluidic system of claim 1, further comprising optical components for characterizing content of droplets.

8. The microfluidic system of claim 1, wherein desired droplets are ejected from the second fluidic network into wells of a microwell plate for downstream processing.

9. The microfluidic system of claim 1, wherein the set of reservoirs comprises magnetic beads.

10. The microfluidic system of claim 1, wherein the incubation region comprises an incubation chamber.

11. The microfluidic system of claim 1, wherein the incubation region comprises an incubation loop.

12. The microfluidic system of claim 11, further comprising a first actuator situated proximate to a first junction between the incubation loop and the channel, and a second actuator proximate to a second junction between the incubation loop and the channel.

13. The microfluidic system of claim 12, wherein the first actuator comprises a first resistor, and wherein the second actuator comprises a second resistor.

14. The microfluidic system of claim 1, further comprising one or more magnetic actuation regions downstream of the incubation region.

15. The microfluidic system of claim 14, further comprising a separation region downstream of the one or more magnetic actuation regions, the separation region comprising one or more side channels.

16. The microfluidic system of claim 1, further comprising, downstream of the incubation region, a washing reagent reservoir and an elution reagent reservoir.

17. The microfluidic system of claim 1, further comprising a recycling loop having an inlet situated downstream of the incubation region, and an outlet situated upstream of the set of reservoirs.

18. The microfluidic system of claim 1, further comprising one or more actuators to drive fluids flow through the set of independent fluidic networks.

19. A method for selecting cells producing a specific product using a microfluidic device, the method comprising:
packaging, into droplets, via a first fluidic network of the microfluidic device, cells that are in a cell suspension, wherein the cells are packaged into the droplets such that each droplet includes (1) no more than one cell from the cell suspension, and (2) one or more reagents from one or more reagent reservoirs of the microfluidic device;
ejecting droplets into an incubation region of the microfluidic device;
incubating the droplets in the incubation region at a temperature and for a period of time;
pulling incubated droplets from the incubation region into a second fluidic network of the microfluidic device;
detecting a desired product in a subset of the incubated droplets pulled from the incubation region; and
sorting the incubated droplets based on whether the desired product is detected in the incubated droplets.

20. The method of claim 19, wherein each cell includes a nucleic acid encoding for production of a different product.
